SEQUENCE LISTING (24 sequences)

SEQ ID NO: 1 - protein, 504 aa, synthetic construct
  REGION 1..504: note = synthetic sequence
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 2 - protein, 340 aa, synthetic construct
  REGION 1..340: note = synthetic sequence
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNER

SEQ ID NO: 3 - protein, 120 aa, synthetic construct
  REGION 1..120: note = synthetic sequence
EVQLVQSGAEVKKPGESLRISCKASGYTFTSYWVHWVRQMPGKGLEWIGNIDPSDSDTHYNQKFKDHVTLSVDKSISTAYLQLSSLKASDTAMYYCVRGGTGTMAWFAYWGQGTLVTVSS

SEQ ID NO: 4 - protein, 120 aa, synthetic construct
  REGION 1..120: note = synthetic sequence
EVQLVQSGAEVKKPGESLRISCKASGYSFTSYWVHWVRQMPGKGLEWMGNIDPSDSDTHYNQKFKDHVTLSVDKSISTAYLQLSSLKASDTAMYYCVRGGTGTMAWFAYWGQGTLVTVSS

SEQ ID NO: 5 - protein, 120 aa, synthetic construct
  REGION 1..120: note = synthetic sequence
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTMAWFAYWGQGTLVTVSS

SEQ ID NO: 6 - protein, 120 aa, synthetic construct
  REGION 1..120: note = synthetic sequence
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAWFAYWGQGTLVTVSS

SEQ ID NO: 7 - protein, 120 aa, synthetic construct
  REGION 1..120: note = synthetic sequence
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTMAYFAYWGQGTLVTVSS

SEQ ID NO: 8 - protein, 120 aa, synthetic construct
  REGION 1..120: note = synthetic sequence
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSS

SEQ ID NO: 9 - protein, 112 aa, synthetic construct
  REGION 1..112: note = synthetic sequence
DVVMTQSPLSLPVTLGQPASISCRSSQSLVHSYGNTYLYWYQQRPGQSPRLLIYRVSNRFSGVPDRFSGSGSGTDFTLKISRVEAEDVGVYFCFQGTHVPYTFGGGTKVEIK

SEQ ID NO: 10 - protein, 112 aa, synthetic construct
  REGION 1..112: note = synthetic sequence
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIK

SEQ ID NO: 11 - protein, 447 aa, synthetic construct
  REGION 1..447: note = synthetic sequence
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 12 - protein, 219 aa, synthetic construct
  REGION 1..219: note = synthetic sequence
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, synthetic construct
  REGION 1..446: note = synthetic sequence
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 14 - protein, 1152 aa, synthetic construct
  REGION 1..1152: note = synthetic sequence
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 15 - protein, 5 aa, synthetic construct
  REGION 1..5: note = synthetic sequence
SYWVH

SEQ ID NO: 16 - protein, 16 aa, synthetic construct
  REGION 1..16: note = synthetic sequence
NIDPSDSDTHYNQKFK

SEQ ID NO: 17 - protein, 16 aa, synthetic construct
  REGION 1..16: note = synthetic sequence
NIDPSDSDTHYSPSFQ

SEQ ID NO: 18 - protein, 11 aa, synthetic construct
  REGION 1..11: note = synthetic sequence
GGTGTMAWFAY

SEQ ID NO: 19 - protein, 11 aa, synthetic construct
  REGION 1..11: note = synthetic sequence
GGTGTLAWFAY

SEQ ID NO: 20 - protein, 11 aa, synthetic construct
  REGION 1..11: note = synthetic sequence
GGTGTMAYFAY

SEQ ID NO: 21 - protein, 11 aa, synthetic construct
  REGION 1..11: note = synthetic sequence
GGTGTLAYFAY

SEQ ID NO: 22 - protein, 16 aa, synthetic construct
  REGION 1..16: note = synthetic sequence
RSSQSLVHSYGNTYLY

SEQ ID NO: 23 - protein, 7 aa, synthetic construct
  REGION 1..7: note = synthetic sequence
RVSNRFS

SEQ ID NO: 24 - protein, 9 aa, synthetic construct
  REGION 1..9: note = synthetic sequence
FQGTHVPYT

(19) United States
(12) Patent Application Publication
(10) Pub. No.: US 2026/0358342 A1
(43) Pub. Date: Nov. 12, 2026

(54) Predictive Efficacy Biomarkers for Anti-SIRPa Antibodies

(71) Applicants: BOEHRINGER INGELHEIM INTERNATIONAL GMBH, Ingelheim am Rhein (DE); OSE IMMUNOTHERAPEUTICS, Nantes (FR)

(72) Inventors: Isabelle Girault, Nantes (FR); Thomas Vandewalle, Nantes (FR); Mabrouk Elgadi, Ridgefield, CT (US)

(21) Appl. No.: 19/130,002

(22) PCT Filed: Nov. 16, 2023
(86) PCT No.: PCT/EP2023/082095

§ 371 Date: May 14, 2025

(30) Foreign Application Priority Data
Nov. 16, 2022 (EP) 22306687.9

(51) Int. Cl. G01N 33/575; A61K 39/395; C07K 16/28

(52) U.S. Cl. CPC G01N 33/5758; A61K 39/3955; C07K 16/2803; G01N 2333/70503; G01N 2333/70553

(57) ABSTRACT

The application generally relates to the treatment of cancer in patients who exhibit immune cells, in particular myeloid cells, for example Tumor Associated Macrophages, Monocytes, myeloid dendritic cells, MDSCs, and/or Tumor Associates Neutrophils, that express the biological markers CD11b and SIRPα in the microenvironment of a tumor. These patients have been identified as the best responders to therapies that comprise the administration of an anti-SIRPα antagonist antibody.

### FIELD OF THE INVENTION

The application generally relates to the treatment of patients having a cancer by using an anti-SIRPα antibody or antigen-binding fragment thereof. In particular, the present invention relates to uses and methods of using anti-SIRPα antagonist antibody or antigen-binding fragment thereof to treat patients who are more likely to positively respond to a treatment against cancer by administering an anti-SIRPα antibody or antigen-binding fragment thereof, alone or in combination with another therapeutic compound (like another antibody) or therapeutic method (like radiotherapy or chemotherapy) or a so-called Standard of Care treatment (i.e., the therapy usually recommended to treat the cancer occurring in the patient).

The application thus relates to the treatment of cancer in patients who exhibit immune cells, in particular myeloid cells, in particular Tumor Associated Macrophages, Monocytes, myeloid dendritic cells, Tumor Associates Neutrophils and/or MDSCs, more particularly Myeloid-derived suppressor cells (MDSCs), that express the biological markers CD11b and SIRPα in the microenvironment of a tumor. These patients have been identified according to the invention as the best responders to therapies that comprises the administration of an anti-SIRPα antagonist antibody. The invention is more particularly dedicated to the measurement of these particular markers expressed by immune cells, in particular myeloid cells, for example Tumor Associated Macrophages, Monocytes, myeloid dendritic cells, Tumor Associates Neutrophils and/or MDSCs, obtained from the patient having a cancer, administering to the patients who have cells that express the particular markers an anti-SIRPα antibody or antigen-binding fragment thereof, either alone or in combination.

### BACKGROUND OF THE INVENTION

Cancer is a major worldwide health concern causing the death of approximately 9.5 million people a year, while more than 20 million people develop a cancer within a year (world cancer report by World Health Organization, 2018). Targeting immune checkpoints of the adaptive immunity has shown great therapeutic efficacy to fight numerous cancers. Immune checkpoints on myeloid cells like macrophages, dendritic cells (DC), myeloid-derived suppressor cells (MDSCs), and polymorphonuclear leukocytes or neutrophils (PMN), remain poorly studied while these cells represent an abundant immune cell type in many solid tumors and are often associated with a poor outcome. Signal regulatory protein alpha (SIRPα, also designated SIRP-alpha, SIRPα, CD172a or SHPS-1), is expressed on monocytes, most subpopulations of tissue macrophages, MDSCs, granulocytes, subset of DC in lymphoid tissues, some bone marrow progenitor cells, and to varying levels on neurons, with a notable expression in synapse-rich areas of the brain, such as the granular layer of the cerebellum and the hippocampus.

SIRPα is the prototypic member of the SIRP paired receptor family of closely related SIRP proteins comprising SIRPα, SIRPg (also designated SIRP-gamma, SIRPγ, CD172g or SIRP beta-2) and SIRPb (also designated SIRP-beta, SIRPB, CD172b). Signal regulatory proteins (SIRPs) constitute a family of cell surface glycoproteins which are expressed on myeloid (including macrophages, granulocytes, myeloid dendritic cells, and mast cells) and neuronal cells (summarized in Barclay, A. N. & Brown, M. H., Nat Rev Immunol 6, 457-64 (2006); see also WO 97/48723) as well on some normal tissue cells and tumor cells. CD47, a broadly expressed transmembrane glycoprotein, functions as a cell-bound ligand for SIRPα and binds to the NH2-terminal extracellular terminus of SIRPα. SIRPα's role has been best documented in respect of its inhibitory role in the phagocytosis of host cells by macrophages. In particular, the binding of SIRPα on macrophages by CD47 expressed on target cells, generates an inhibitory signal that negatively regulates phagocytosis. However, more recent findings have also demonstrated additional positive signaling effects mediated through SIRPα binding (Shultz, L. D. et al. (1995) J Immunol 154, 180-91).

Expressed by myeloid cells, SIRPα interacts with the ubiquitous receptor CD47, and this interaction is an important immune checkpoint of the innate response, involved in the regulation of myeloid functions. The interaction between SIRPα and CD47 provides a down-regulatory signal that inhibits host cell phagocytosis. Since CD47 is widely overexpressed in some cancer cells, CD47 functions as a “don't eat me” signal within some tumor comprising these cells, thereby avoiding phagocytosis. The potential contribution of CD47-SIRPα interaction in cancer cell clearance has been intensely investigated in recent years. It was shown that abundance of CD47 receptors in tumors is inversely correlated with patient overall survival and constitutes an adverse prognostic factor for several cancer types.

The SIRPα/CD47 pathway has therefore been subject to different pharmaceutical developments to enhance macrophages phagocytosis. These encompass the use of fragmented/truncated SIRPα and/or CD47 proteins and antibodies thereto. Over-expression of CD47 by cancer cells renders them resistant to macrophages, even when these cells are coated with therapeutic antibodies. The blockade of SIRPα/CD47 pathway via agents targeting CD47 has shown to enhance the antibody-dependent phagocytosis by macrophages. These therapies have also been described to synergize with depleting therapeutic anticancer antibodies such as Trastuzumab (anti-Her2), Cetuximab (anti-EGFR), Rituximab (anti-Cd20) and Alemtuzumab (anti-CD52).

Therefore, anti-human SIRPα antibodies able to disrupt the binding between SIRPα and CD47 have been developed in recent years. There is nonetheless a need for improved used of these antibodies, in particular for tuning their effect in vivo, for example by enhancing the patient response to the anti-SIRPα antibody or antigen-binding fragment thereof. There is also a need for improved used of these compounds in combination with current treatments or current therapies.

In this context, being able to determine, in a reliable manner, the likeliness to positively respond to a treatment to a given patient, and more particularly in a patient to be treated against cancer, is of crucial importance to the patient. In particular, there is a need to assess if the contemplated treatment for a patient having cancer is adapted to effectively treat the disease. WO2014/186761 describes genetic biomarkers associated with responsiveness to anti CD47 agents (and notably anti-SIRPα antibodies) and point out genes SPP1, CHITI, FCyR2A and FCyR3A as putative markers of the responsiveness. WO2020/107115 describes biomarkers for CD47 tumors treatment. Identified markers are secreted proteins or cellular immune receptors (Monocyte Chemoattractant Protein 3 (MCP-3), Monocyte Chemoattractant Protein 1 (MCP-1), Interleukin-1 alpha (IL-1A), Interleukin 8 (IL-8), Macrophage Inflammatory Protein 1-alpha (MIP-1α), Macrophage Inflammatory Protein 1-beta (MIP-1β), Monokine Induced by Gamma Interferon (MIG). These biomarkers are not associated with the effectiveness of a cancer therapy. Besides, the biomarkers described in the above referenced applications do not refer to predictive biomarkers of efficacy. The biomarkers are representative of drug target engagement and their association with clinical response.

Usually, when seeking biomarkers for immunotherapy, the one skilled in the art measures the expression of the target molecule on the tumor to estimate the potential efficiency of the antibodies which target this molecule. The one skilled in the art would thus measure the level of expression of CD47, as it is a molecule expressed by tumor cells to assess if using an anti-SIRPα antibody is likely to benefit to a patient receiving such an antibody as therapy. The inventors have shown that CD47 expression is very heterogeneous in tumor cells.

The applicant has now shown that heterogeneous CD47 expression in tumor cells is observed in progressive disease patients. Besides CD47 has different ligands also involved in tumorigenesis. Accordingly, CD47 would not constitute a reliable predictive biomarker for the treatment of cancer in patients in need thereof when the treatment is based on the use of anti-SIRPα antibodies or antigen-binding fragment thereof.

### SUMMARY OF THE INVENTION

The inventors have developed a new approach that comprises assessment of patients' status with respect to biomarkers that would exhibit predictive value relating to the benefit of a cancer treatment encompassing anti-SIRPα antibodies or antigen-binding fragment thereof wherein the assessment is not based on the consequence of the blockage of CD47/SIRPα axis, but on the level of expression of specific immune cells, namely on CD11b+/SIRPα+ myeloid cells in the tumor microenvironment, for example in Tumor Associated Macrophages, Monocytes, myeloid dendritic cells, Tumor Associates Neutrophils and/or MDSCs, in particular in MDSCs.

There is a higher level of CD11b⁺ myeloid cells expressing SIRPα for stable disease and partial response patients compared to progressive disease patients at baseline (baseline=before treatment with the anti-SIRPα antibody). High levels of CD11b⁺ myeloid cells expressing SIRPα is the driver of a better overall survival rather than CD47.

As it will be shown in the application, the levels of CD11b+ myeloid cells also expressing SIRPα, in the patient's tumor microenvironment (TME) is associated with a better Overall Survival (OS) and consequent drug efficacy. The level of CD11b+/SIRPα+ myeloid cells is accordingly provided as a key predictive biomarker for patient eligibility for treatment with anti-SIRPα antibodies or antigen-binding fragment thereof. The applicant has accordingly shown that the level of CD11b⁺ myeloid cells also expressing SIRPα in the TME will determine the drug efficacy.

As a conclusion CD11b⁺ myeloid cells also expressing SIRPα from tumor microenvironment is much more relevant than CD47 expression on tumor cells to select patients with better chance to benefit from anti SIRPα antibodies or antigen binding fragments thereof. Furthermore, the potential of anti SIRPα antibodies or antigen binding fragments thereof is best correlated to the expression of SIRPα by CD11b+ myeloid cells. Concurrently, the expression of SIRPα on tumor cells was not found to be associated with a better Overall Survival (OS) and consequent drug efficacy.

The invention hence provides new uses and methods of treating cancer in a patient comprising administering an anti-SIRPα compound, in particular an anti-SIRPα antibody or antigen-binding fragment thereof. Stated otherwise, the invention provides uses of an anti-SIRPα compound, in particular an anti-SIRPα antibody and antigen-binding fragment thereof, and methods wherein such compounds are administered to a patient having a cancer, when the patient has been shown to likely benefit or positively respond to such a treatment.

The inventors herein provide uses and administering methods of an anti-SIRPα compound, in particular an anti-SIRPα antibody and antigen-binding fragment thereof, in a monotherapy, or in a combination therapy (either simultaneously, subsequently or as a primo-therapy) with other therapeutic agents (like but not limited to immuno-agents, like immune checkpoint inhibitors or activators) and/or therapeutic methods (like but not limited to surgery, radiotherapy, chemotherapy, hormonotherapy).

As it will be explained in detail in the present application, at least two major advances have been obtained. According to a first aspect, the inventors of the present invention have identified that patients diagnosed with cancer who have a tumor whose microenvironment (TME) that comprises myeloid cells expressing both CD11b and SIRPα are more likely to benefit from a treatment of their cancer, e.g. to show an increase overall survival than expected without treatment or to positively respond to a treatment of their cancer when administered an anti-SIRPα agent, in particular anti-SIRPα antibodies or antigen-binding fragment thereof that inhibit the binding between CD47 and SIRPα.

According to a particular embodiment, the patients diagnosed with cancer have at least 55%, in particular at least 60%, in particular at least 65.3% of their myeloid cells in the microenvironment of the tumor expressing the biological markers CD11b and SIRPα are more likely to positively respond to a treatment of their cancer by the administration of an anti-SIRPα agent, in particular anti-SIRPα antibodies or antigen-binding fragment thereof that inhibit the binding between CD47 and SIRPα. While it was previously thought that CD47 should be measured to assess if a therapy based on the inhibition of the CD47-SIRPα interaction is likely to succeed, since CD47 is usually expressed by tumor cells, the inventors show in a first aspect of the present invention that overall survival correlates with SIRPα expression in myeloid cells expressing CD11b (p-value <0.05).

The level of myeloid cells, in particular Tumor Associated Macrophages, Monocytes, myeloid dendritic cells, Tumor Associates Neutrophils and/or MDSCs, more particularly MDSCs, expressing the biomarkers according to the invention is representative of measurement performed with ImmunoHistoChemistry. Other methods for characterizing markers expressed by cells can be used to measure the level of myeloid cells, in particular Tumor Associated Macrophages, Monocytes, myeloid dendritic cells, Tumor Associates Neutrophils and/or MDSCs, more particularly MDSCs, expressing the biomarkers according to the invention.

Usually, determining if a cancer therapy is likely to succeed requires determining or measuring expression of biological markers by tumor cells, and not on myeloid cells of the patient. The inventors found that a positive clinical outcome or response is observed in patients having at least 55%, in particular at least 60%, in particular at least 65.3% of CD11b-positive and SIRPα-positive myeloid cells, as compared to having less than 60%, in particular less than 65.3%, of CD11b-positive and SIRPα-positive myeloid cells. The percentage of myeloid cells expressing CD11b and SIRPα is associated with a better overall survival (OS) of patients treated with an anti-SIRPα antagonist antibody (either in monotherapy or in combination therapy), and a better efficacy of the therapy. CD11b-positive and SIRPα-positive myeloid cells (also referenced CD11+/SIRPα+ myeloid cells) is thus a key predictive biomarker for selecting or targeting patients who are likely to benefit from a therapy comprising the administration of an anti-SIRPα antagonist antibody or antigen-binding fragment thereof.

In a particular embodiment, the myeloid cells expressing CD11b and SIRPa in the TME are measured before administering a treatment to the patient, in particular before administering an anti-SIRPα antibody or an antigen-binding fragment thereof that that inhibits the binding between SIRPα and CD47 to the patient. The measure of the myeloid cells expressing CD11b and SIRPa in the Tumor microenvironment (TME) before treatment is then considered at the baseline level of CD11b+ and SIRPα+ myeloid cells.

In a particular embodiment, the myeloid cells expressing CD11b and SIRPa in the TME are measured during the administration of a treatment to the patient, in particular during the administration of an anti-SIRPα antibody or an antigen-binding fragment thereof that that inhibits the binding between SIRPα and CD47 to the patient. The measure of the myeloid cells expressing CD11b and SIRPa in the Tumor microenvironment (TME) during the treatment is then considered at the baseline level of CD11b+ and SIRPα+ myeloid cells. During the treatment means that several administrations are scheduled (at least two), the measurement(s) being performed along with or after the first administration and before the last scheduled administration).

It is provided a composition comprising an anti-SIRPα compound, in particular an anti-SIRPα antibody or an antigen-binding fragment, thereof that inhibits the binding between SIRPα and CD47, preferably between human SIRPα and human CD47, for use in the treatment of cancer in a patient, wherein the cancer is characterized by a tumor whose microenvironment (TME) comprises myeloid cells, in particular Tumor Associated Macrophages, Monocytes, myeloid dendritic cells, Tumor Associates Neutrophils and/or MDSCs, more particularly MDSCs, that express both CD11b and SIRPα.

In a particular embodiment, it is provided a composition comprising an anti-SIRPα compound, in particular an anti-SIRPα antibody or an antigen-binding fragment thereof that that inhibits the binding between SIRPα and CD47, preferably between human SIRPα and human CD47, for use in the treatment of cancer in a patient, wherein the cancer is characterized by a tumor whose microenvironment (TME) comprises Tumor Associated Macrophages, Monocytes, myeloid dendritic cells, Tumor Associates Neutrophils and/or MDSCs that express both CD11b and SIRPα. Each of Tumor Associated Macrophages, Monocytes, myeloid dendritic cells, Tumor Associates Neutrophils and/or MDSCs may be considered as a subgroup of myeloid cells.

In a particular embodiment, the composition is for use in the treatment of cancer in a patient, wherein the cancer is characterized by a tumor whose microenvironment (TME) wherein at least 55%, in particular at least 60%, preferably at least 65.3% of myeloid cells, in particular Tumor Associated Macrophages, Monocytes, myeloid dendritic cells, Tumor Associates Neutrophils and/or MDSCs, more particularly MDSCs, present within the microenvironment of the tumor express the biological markers CD11b and SIRPα when tested in ImmunoHistoChemistry (IHC).

In a further embodiment of the described use, the microenvironment of the tumor of the patient comprises Tumor Associated Macrophages, Monocytes, myeloid dendritic cells, Tumor Associates Neutrophils and/or MDSCs that express the biological markers CD11b and SIRPα.

In a further embodiment of the described use, the microenvironment of the tumor of the patient comprises Tumor Associated Macrophages, that express the biological markers CD11b and SIRPα.

In a further embodiment of the described use, the microenvironment of the tumor of the patient comprises Monocytes that express the biological markers CD11b and SIRPα.

In a further embodiment of the described use, the microenvironment of the tumor of the patient comprises myeloid dendritic cells that express the biological markers CD11b and SIRPα.

In a further embodiment of the described use, the microenvironment of the tumor of the patient comprises Tumor Associates Neutrophils that express the biological markers CD11b and SIRPα.

In a further embodiment of the described use, the microenvironment of the tumor of the patient comprises Tumor Associated Macrophages, Monocytes, myeloid dendritic cells, Tumor Associates Neutrophils and MDSCs that express the biological markers CD11b and SIRPα.

In a particular embodiment, the patient has myeloid cells within the microenvironment of the tumor, said myeloid cells comprising Tumor Associated Macrophages, Monocytes, myeloid dendritic cells, Tumor Associates Neutrophils and/or MDSCs, in particular MDSCs.

In a particular embodiment, it is provided a composition comprising an anti-SIRPα compound, in particular an anti-SIRPα antibody or an antigen-binding fragment, thereof that inhibits the binding between SIRPα and CD47, preferably between human SIRPα and human CD47, for use in the treatment of a patient who has a cancer, the patient having:
at least 55%, in particular at least 60%, in particular at least 65.3% of its myeloid cells present within the microenvironment of a tumor, in particular Tumor Associated Macrophages, Monocytes, myeloid dendritic cells, Tumor Associates Neutrophils and/or MDSCs, that express the biological markers CD11b and SIRPα. Accordingly, the invention also relates to a composition comprising an anti-SIRPα antibody or an antigen-binding fragment thereof that inhibits the binding between SIRPα and CD47, preferably between human SIRPα and human CD47, for use in a method of treating cancer in a patient who has cancer, wherein the method comprises:
   measuring the presence of myeloid cells, in particular Tumor Associated Macrophages, Monocytes, MDSCs, myeloid dendritic cells, and/or Tumor Associates Neutrophils, expressing CD11b and SIRPα in a biological sample obtained from the patient, wherein the myeloid cells are from the microenvironment of a tumor,
   when at least 55%, in particular at least 60%, in particular at least 65.3% of its myeloid cells present within the microenvironment of a tumor, in particular Tumor Associated Macrophages, Monocytes, MDSCs, myeloid dendritic cells, and/or Tumor Associates Neutrophils, that express the biological markers CD11b and SIRPα;
   administering a treatment comprising anti-SIRPα antibody or an antigen-binding fragment thereof that inhibits the binding between SIRPα and CD47, preferably between human SIRPα and human CD47.

In a particular embodiment, the composition for use according to the invention as disclosed in the various embodiments is for the treatment of a liquid cancer or a solid cancer, in particular a cancer with advanced solid tumor(s), more particularly an adrenal cancer, a biliary tree cancer, a breast cancer, a cervical cancer, a colorectal cancer, an endometrial cancer, a gastrointestinal cancer, a head and neck cancer, a kidney cancer, a liver cancer, a lung cancer, a melanoma, a non-small cells lung cancer, an ovarian cancer, a hepatocellular carcinoma, a pancreas cancer, a parotid cancer, a prostate cancer or an uterine cancer; more particularly an ovarian cancer, a breast cancer in particular a Triple Negative Breast cancer, lung cancer, most particularly Non-Small Cell Lung Cancer (NSCLC), cervical cancer, or colorectal cancer, most particularly ovarian cancer, colorectal cancer or Non-Small Cell Lung Cancer (NSCLC).

In a third aspect of the invention, the inventors found that the presence or not of particular immune cells with the determined predictive biomarkers disclosed herein within the microenvironment of a tumor in a patient is associated with respectively a clinical benefit, in particular a positive response or negative response of the patient to a treatment against cancer with an anti-SIRPα antibody or an antigen-binding fragment thereof that inhibits the binding between SIRPα and CD47, in particular in combination with a second therapeutic agent.

It is thus provided a composition comprising an anti-SIRPα antibody or an antigen-binding fragment thereof that inhibits the binding between SIRPα and CD47, preferably between human SIRPα and human CD47is for use in a combination treatment with an immune checkpoint inhibitor of the interaction between tumor cell and myeloid cells, in particular with a compound that inhibits the interaction between PD-1 and PD-L1, preferably between human PD-1 and human PD-L1 such as a therapeutic agent selected from an anti-PD1 or an anti-PD-L1 antagonist antibody or an anti-PD1 antagonist antibody, more preferably an anti-PD-1 antagonist antibody or with a compound that targets the lymphocyte activation gene-3 (LAG-3), in particular an antibody against lymphocyte activation gene-3, for simultaneous or subsequent administration, for use in the treatment of a patient who has a cancer and who is likely to positively respond to said treatment according to the test(s) provided according to the invention.

In a particular embodiment, the patient has Tumor Associated Macrophages, Monocytes, myeloid dendritic cells, Tumor Associates Neutrophils and/or MDSCs within the microenvironment of the tumor.

In a particular embodiment, the patient has Tumor Associated Macrophages, within the microenvironment of the tumor.

In a particular embodiment, the patient has Monocytes within the microenvironment of the tumor.

In a particular embodiment, the patient has myeloid dendritic cells within the microenvironment of the tumor.

In a particular embodiment, the patient has Tumor Associates Neutrophils within the microenvironment of the tumor.

In a particular embodiment, the patient has MDSCs within the microenvironment of the tumor.

In a particular embodiment, the patient has Tumor Associated Macrophages, Monocytes, myeloid dendritic cells, Tumor Associates Neutrophils and MDSCs within the microenvironment of the tumor.

The administration of the anti-SIRPα antibody may lead to a raise of the expression of PD-1 and/or PD-L1 within the tumor. Accordingly, the use in combination of an anti-SIRPα antibody with an anti-PD-L1 antibody or anti-PD-1 antibody may be useful in the treatment of certain types of cancer, in particular cancers than have shown resistance to monotherapy treatment. The anti-SIRPα antibody leads to enhancement of the anti-tumor effect of the anti-PD-L1 antibody or anti-PD-1 antibody, in particular in patients who have shown positive for the CD11b and SIRPα biomarkers according to the invention and optionally who have Tumor Associated Macrophages, Monocytes, myeloid dendritic cells, Tumor Associates Neutrophils and/or MDSCs, in particular MDSCs, within the microenvironment of the tumor.

The use of the anti-SIRPα antibody or antigen-binding fragment thereof as disclosed herein enhances the presence of immune cells within the microenvironment of the tumor. The administration of the anti-SIRPα antibody or antigen-binding fragment may increase the expression of particular markers, like PD-L1, thereby potentiating the use of other therapies in combination with the administration of the anti-SIRPα agent. The inventors have shown that such a combination of therapeutic agents is particularly effective in patients who have Tumor Associated Macrophages, Monocytes, myeloid dendritic cells, Tumor Associates Neutrophils and/or MDSCs, in particular MDSCs, within the microenvironment of the tumor.

According to an aspect of the invention, a method is provided for assessing a patient's status for biomarkers of response to a treatment with an anti-SIRPα antibody or an antigen-binding fragment thereof that inhibits the binding between SIRPα and CD47, preferably between human SIRPα and human CD47, wherein the patient is in need of treatment of a cancer with a solid tumor, the method comprising:
providing a biological sample previously obtained from the patient wherein the sample is from the microenvironment of the patient's tumor (TME) and comprises myeloid cells, in particular the sample is a biopsy of the microenvironment of the patient's tumor,
determining the presence in the biological sample of myeloid cells expressing CD11b and SIRPα biomarkers, in particular of Tumor Associated Macrophages, Monocytes, myeloid dendritic cells, Tumor Associates Neutrophils and MDSCs, more particularly MDSCs, expressing CD11b and SIRPα biomarkers.

According to a particular embodiment, it is also provided a method for determining if a patient having a cancer is likely to benefit from a therapy to treat the cancer, the method comprising:
Providing a biological sample, previously obtained from the patient wherein the sample is from the microenvironment of the patient's tumor (TME) and comprises myeloid cells, in particular the sample is a biopsy of the microenvironment of the patient's tumor,
determining or measuring the presence of myeloid cells, in particular Tumor Associated Macrophages, Monocytes, MDSCs, myeloid dendritic cells, and/or Tumor Associates Neutrophils, expressing CD11b and SIRPα in the biological sample,
if myeloid cells, in particular Tumor Associated Macrophages, Monocytes, myeloid dendritic cells, Tumor Associates Neutrophils and/or MDSCs, expressing CD11b and SIRPα biomarkers are present in the biological sample, the patient is likely to benefit from the treatment.

In a particular embodiment, when the percentage of myeloid cells expressing CD11b and SIRPα is at least 55%, in particular at least 60%, in particular at least 65.3% in the biological sample, classifying the patient as likely to positively respond to a treatment of their cancer by administration of an anti-SIRPα antibody or an antigen-binding fragment thereof that inhibits the binding between SIRPα and CD47, preferably between human SIRPα and human CD47.

According to a particular embodiment, it is also provided a method for discriminating between patients having a cancer, those who are likely to positively respond to a treatment of their cancer by administration of an anti-SIRPα antibody or an antigen-binding fragment thereof that inhibits the binding between SIRPα and CD47, preferably between human SIRPα and human CD47, from those who are not, the method comprising:
Providing a biological sample previously obtained from a patient, wherein the sample is from the microenvironment of the patient's tumor (TME) and comprises myeloid cells, in particular the sample is a biopsy of the microenvironment of the patient's tumor
Determining or measuring the presence of myeloid cells, in particular Tumor Associated Macrophages, Monocytes, MDSCs, myeloid dendritic cells, Tumor Associates Neutrophils, expressing CD11b and SIRPα in the biological sample,
if myeloid cells expressing CD11b and SIRPα biomarkers are present in the biological sample, the patient is likely to benefit from the treatment, and/or the patients are likely to positively respond to the treatment.

In a particular embodiment, the patients who are likely to positively respond to the treatment are those who have at least 55%, in particular at least 60%, in particular 65.3% of their myeloid cells that express CD11b and SIRPα in their biological sample.

According to a particular embodiment, it is also provided a method of treating a cancer in a patient having cancer, the method comprising:
Providing a biological sample previously obtained from a patient, wherein the sample is from the microenvironment of the patient's tumor (TME) and comprises myeloid cells, in particular the sample is a biopsy of the microenvironment of the patient's tumor
Measuring the presence of myeloid cells, in particular Tumor Associated Macrophages, Monocytes, MDSCs, myeloid dendritic cells, and/or Tumor Associates Neutrophils, expressing CD11b and SIRPα in the biological sample,
When myeloid cells expressing CD11b and SIRPα biomarkers are present in the biological sample, administering to the patient an anti-SIRPα antibody or an antigen-binding fragment thereof that inhibits the binding between SIRPα and CD47, preferably between human SIRPα and human CD47, either alone or in a combination treatment with an immune checkpoint inhibitor of the interaction between tumor cell and myeloid cells, in particular with a compound that inhibits the interaction between PD-1 and PD-L1, preferably between human PD-1 and human PD-L1 such as a therapeutic agent selected from an anti-PD1 or an anti-PD-L1 antagonist antibody or an anti-PD1 antagonist antibody, more preferably an anti-PD-1 antagonist antibody or with a compound that targets the lymphocyte activation gene-3 (LAG-3), in particular an antibody against lymphocyte activation gene-3.

In a particular embodiment of this method, the percentage of myeloid cells expressing CD11b and SIRPα is at least 55%, in particular at least 60%, in particular at least 65.3% in the biological sample.

In a particular embodiment of the method of treating a cancer the patient is administered an anti-SIRPα antibody or an antigen-binding fragment thereof that inhibits the binding between SIRPα and CD47, preferably between human SIRPα and human CD47 in a combination treatment with a second therapeutic agent, in particular an anti-PD1 or an anti-PD-L1 antibody.

In a particular embodiment of this method of treatment the patient has liquid or solid cancer, in particular a cancer with advanced solid tumor(s), more particularly an adrenal cancer, a biliary tree cancer, a breast cancer, a cervical cancer, a colorectal cancer, an endometrial cancer, a gastrointestinal cancer, a head and neck cancer, a kidney cancer, a liver cancer, a lung cancer, a melanoma, a non-small cells lung cancer, an ovarian cancer, a hepatocellular carcinoma, a pancreas cancer, a parotid cancer, a prostate cancer or an uterine cancer, more particularly an ovarian cancer, a breast cancer in particular a Triple Negative Breast cancer, lung cancer, most particularly Non-Small Cell Lung Cancer (NSCLC), cervical cancer, or colorectal cancer, most particularly ovarian cancer, colorectal cancer or Non-Small Cell Lung Cancer (NSCLC).

According to particular embodiments, the present invention provides an anti-SIRPα antibody or antigen-binding fragment thereof for use in the treatment of cancer with solid tumor(s), more particularly cancer with advanced solid tumor(s), more particularly an adrenal cancer, a biliary tree cancer, a breast cancer, a cervical cancer, a colorectal cancer, an endometrial cancer, a gastrointestinal cancer, a head and neck cancer, a kidney cancer, a liver cancer, a lung cancer, a melanoma, a non-small cells lung cancer, an ovarian cancer, a hepatocellular carcinoma, a pancreas cancer, a parotid cancer, a prostate cancer or an uterine cancer, more particularly an ovarian cancer, a breast cancer in particular a Triple Negative Breast cancer, lung cancer, most particularly Non-Small Cell Lung Cancer (NSCLC), cervical cancer, or colorectal cancer, wherein the patient is identified for exhibiting the biomarkers disclosed according to the invention.

According to particular embodiments, the present invention provides an anti-SIRPα antibody or antigen-binding fragment thereof for use in combination with an immune checkpoint inhibitor or activator in the treatment of cancer, wherein the patient is identified for exhibiting the predictive biomarkers disclosed according to the invention.

According to particular embodiments, the present invention provides an anti-SIRPα antibody or antigen-binding fragment thereof for use in the treatment of patients who, prior to the use, were known or shown not to respond to a cancer treatment or therapy and/or exhibited disease progression of a cancer despite being under treatment. According to this embodiment, the invention provides an anti-SIRPα SIRPα antibody or antigen-binding fragment thereof for use in the treatment of cancer in a patient who has failed treatment with at least one alternative cancer therapeutic agent or therapeutic method, wherein the patient is identified for exhibiting the predictive biomarkers disclosed according to the invention.

According to particular embodiments, the present invention provides an anti-SIRPα antibody or antigen-binding fragment thereof for use in the treatment of patients who, prior to the use, has not yet been treated. According to this embodiment, the invention provides an anti-SIRPα antibody or antigen-binding fragment thereof for use in the treatment of cancer in a patient as a first therapy. According to particular embodiments, the present invention provides an anti-SIRPα SIRPa antibody or antigen-binding fragment thereof for use in the treatment of patients who have been treated with immune checkpoint inhibitors or activators, in particular with an anti-PD-1 or an anti-PD-L1 agent, and did not positively respond or did not maintain response to the administration of the immune checkpoint inhibitors or activators (i.e. the patients show disease progression and/or do not show disease regression), wherein the patient is identified for exhibiting the biomarkers disclosed according to the invention.

### DETAILED DESCRIPTION OF THE INVENTION

### Definitions

As used herein, the term “antibody” refers to any kind of antibodies, such as monoclonal antibodies, polyclonal antibodies, recombinant antibodies, chimeric antibodies and humanized antibodies. The term “antibody” may also refer to deimmunized antibodies, i.e. antibodies wherein T-cell epitopes have been removed from the structure of the antibodies without significantly reducing the binding affinity of the antibody of its target SIRPα. Typically, “deimmunized” antibodies are created with human constant regions. The antibodies of the present invention include monoclonal and polyclonal antibodies. As used herein, a “monoclonal antibody” is intended to refer to a preparation of antibody molecules, antibodies which share a common heavy chain and common light chain amino acid sequence, in contrast with “polyclonal” antibody preparations which contain a mixture of antibodies of different amino acid sequence. Monoclonal antibodies can be generated by several known technologies like phage, bacteria, yeast or ribosomal display, as well as by classical methods exemplified by hybridoma-derived antibodies. Thus, the term “monoclonal” is used to refer to all antibodies derived from one nucleic acid clone. The antibodies of the present invention include recombinant antibodies. As used herein, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies. The antibodies of the present invention include chimeric antibodies. As used herein, a “chimeric antibody” refers to an antibody in which the sequence of the variable domain derived from the germline of a mammalian species, such as a mouse, have been grafted onto the sequence of the constant domain derived from the germline of another mammalian species, such as a human. The antibodies of the present invention include humanized antibodies. As used herein, a “humanized antibody” refers to an antibody in which CDR sequences derived from the germline of another mammalian species, such as a mouse, have been grafted onto human framework sequences.

As used herein, an “antigen-binding fragment of an antibody” means a part of an antibody, i.e. a molecule corresponding to a portion of the structure of the antibody of the invention, that exhibits antigen-binding capability for SIRPα, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capability can be determined by measuring the affinity between the antibody and the target fragment. These antigen-binding fragments may also be designated as “functional fragments” of antibodies. Antigen-binding fragments of antibodies are fragments which comprise their hypervariable domains designated CDRs (Complementary Determining Regions) or part(s) thereof encompassing the recognition site for the antigen, i.e. the extracellular domain of SIRPα, thereby defining antigen recognition specificity. Antigen binding fragments of an antibody that contain the variable domains comprising the CDRs of said antibody encompass Fv, dsFv, scFv, Fab, Fab′, F(ab′)2. Fv fragments consist of the VL and VH domains of an antibody associated together by hydrophobic interactions; in dsFv fragments, the VH:VL heterodimer is stabilised by a disulphide bond; in scFv fragments, the VL and VH domains are connected to one another via a flexible peptide linker thus forming a single-chain protein. Fab fragments are monomeric fragments obtainable by papain digestion of an antibody; they comprise the entire L chain, and a VH-CH1 fragment of the H chain, bound together through a disulfide bond. The F(ab′)2 fragment can be produced by pepsin digestion of an antibody below the hinge disulfide; it comprises two Fab′ fragments, and additionally a portion of the hinge region of the immunoglobulin molecule. The Fab′ fragments are obtainable from F(ab′)2 fragments by cutting a disulfide bond in the hinge region. F(ab′)2 fragments are divalent, i.e. they comprise two antigen binding sites, like the native immunoglobulin molecule; on the other hand, Fv (a VHVL dimmer constituting the variable part of Fab), dsFv, scFv, Fab, and Fab′ fragments are monovalent, i.e. they comprise a single antigen-binding site. These basic antigen-binding fragments of the invention can be combined together to obtain multivalent antigen-binding fragments, such as diabodies, tribodies or tetrabodies. These multivalent antigen-binding fragments are also part of the present invention.

A composition may refer in particular to a pharmaceutical composition. Such a composition may comprise pharmaceutical acceptable components, like but not limited to pharmaceutically suitable excipient or carrier or vehicle, when used for systemic or local administration. A pharmaceutically suitable carrier or vehicle refers to a non-toxic solid, semi-solid or liquid filler, diluent, encapsulating material and formulation like phosphate buffered saline solutions, distilled water, emulsions such as oil/water emulsions, wetting agents and the like, dextrose, saline, ethanol and combinations thereof.

As used herein, the term “SIRPα” relates to the Signal Regulation Peptide alpha from a mammal species, preferably a human SIRPα. SIRPα is also referenced CD172a, Tyrosine-protein phosphatase non-receptor type substrate 1, SHPS-1. SIRPα is an immunoglobulin-like cell surface receptor for CD47. SIRPα SIRPα acts as docking protein and induces translocation of PTPN6, PTPN11 and other binding partners from the cytosol to the plasma membrane. SIRPα mediates negative regulation of phagocytosis, mast cell activation and dendritic cell activation. CD47 binding with SIRPα prevents maturation of immature dendritic cells and inhibits cytokine production by mature dendritic cells. SIRPα may correspond to the protein referenced under Uniprot No. P78324. Alternatively, SIRPα may correspond to a protein having the amino acid sequence of SEQ ID No. 1. The extracellular domain of SIRPα, which is likely to be recognized and bound to by anti-SIRPα antibodies or antigen-binding fragments thereof agent used in the invention may correspond to the amino acid sequence of SEQ ID No. 2.

As used herein, the term “CD11b” refers to cluster of differentiation molecule 11B. It is also known under the names integrin alpha M (ITGAM), macrophage-1 antigen (Mac-1) or complement receptor 3 (CR3). ITGAM is also known as CR3A. CD11b is one protein subunit that forms heterodimeric integrin alpha-M beta-2 (αMβ2) molecule. CD11b is used to identify myeloid cells, in particular Tumor Associated Macrophages, Monocytes, myeloid dendritic cells, Tumor Associates Neutrophils and/or MDSCs, more particularly MDSCs. Human CD11b can have the amino acid sequence associated with UniProt reference P11215 or may correspond to the amino acid sequence of SEQ ID No. 14.

The terms “cancer” and “tumor” have their general meaning in the art and refer to a group of diseases involving abnormal cell growth with the potential to invade or spread to other parts of the body. The term “cancer” further encompasses both primary and metastatic cancers. A cancer is a disease involving abnormal cell growth with the potential to invade or spread to other parts of the body. According to the invention, the cancer which affects or affected a patient may be selected from the list consisting of bladder cancer, bone cancer, brain cancer, breast cancer, including Triple-Negative Breast cancer, cervical cancer, colon cancer, endometrical cancer, esophageal cancer, gastric cancer, head & neck cancers, hepatocellular carcinoma, hodgkin's lymphoma leukemia, liver cancer, including a non-small cells lung cancer, lung cancer, melanoma, mesothelioma, multiple myeloma myelodysplastic syndrome, non-hodgkin's lymphoma, ovarian cancer, pancreatic cancer, prostate cancer, rectal cancer, renal cancer, sarcoma, skin cancer, testicular cancer, thyroid cancer, an adrenal cancer, a biliary tree cancer, a colorectal cancer, a gastrointestinal cancer, a kidney cancer, a parotid cancer, or an uterine cancer, most particularly an ovarian cancer, a breast cancer, in particular a Triple Negative Breast cancer, a liver cancer, a hepatocellular carcinoma, an endometrial cancer or uterine cancer, most particularly Non-Small Cell Lung Cancer (NSCLC), cervical cancer, or colorectal cancer. In a particular embodiment, the cancer which affects a patient is a breast cancer, ovarian cancer, liver cancer, endometrial cancer, or hepatocellular carcinoma. The microenvironment of the tumor is the ecosystem that surrounds a tumor inside the body. It includes immune cells, the extracellular matrix, blood vessels and other cells, like fibroblasts. The microenvironment of the tumour comprises the space surroundings the tumor, and includes the surrounding blood vessels, immune cells, fibroblasts, signaling molecules and the extracellular matrix. The tumor and the surrounding microenvironment are closely related and interact constantly. Immune cells in the microenvironment can affect the growth and evolution of cancerous cells.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence or relapse of the disease, delaying or slowing the progression of the disease, ameliorating the disease state, providing a remission (partial or total) of the disease, enabling to decrease the administered dose of one or more other medications required or used to treat the disease, increasing the quality of life, enabling progression-free survival (PFS) increasing time to disease progression and/or prolonging survival, in particular overall survival (OS), preventing or alleviating side-effects of current treatment, or treatments that will be developed.

The term “effective dose” or “effective dosage” or “effective amount” or “therapeutically effective dose” is defined as an amount sufficient to achieve or at least partially achieve the desired effect. The term “effective dose” is meant to encompass an amount sufficient to cure or at least partially arrest the disease and its complications or alleviate the symptoms of the disease in a patient already suffering from the disease. Amounts or doses effective for this use will depend on the condition to be treated, the delivered antibody construct, the therapeutic context and objectives, the severity of the disease, prior therapy, the patient's clinical history and response to the therapeutic agent, the route of administration, the size (body weight, body surface or organ size) and/or condition (the age and general health) of the patient, and the general state of the patient's own immune system. The proper dose can be adjusted such that it can be administered to the patient once or over a series of administrations, and in order to obtain the optimal therapeutic effect. In a particular embodiment, an effective dose is a dose that enables a decrease in the volume of the tumor (or a tumor shrinkage), or an absence of progression of the volume of the tumor in the treated patient. As an example, a “therapeutically acceptable amount” of a therapeutic agent, like an antibody or an antigen-binding fragment thereof, may be comprised between about 0.1 mg and about 50 mg per kg of weight of the patient (agent/body weight of the patient).

A “marker” (or biomarker) is defined as a biochemical, molecular, or cellular alteration that is measurable in biological tissue such as tissues, cells, or fluids, and that indicates, e.g., is functionally related to normal or abnormal process of a condition or disease. The term “biomarker” refers to molecule which can be measured accurately and reproducibly, thereby leading to the provision of a “signature” that is objectively measured and evaluated as an indicator of normal biological processes, or pathogenic processes, or pharmacologic responses. In the context of the present invention, a biomarker corresponds to biological molecule(s) expressed by and/or present within cells of a human being. Thus, in the present invention biological markers include protein biomarkers, genetic biomarkers (corresponding to the transcript products of genes) and epigenetic biomarker (corresponding to methylation of DNA for example). In the present invention, biomarkers include DNA, RNA and proteins. CD11b and SIRPα are considered as markers in the context of the present invention.

The patient may be any human who had or is suspected to have or who develops a cancer. In particular, the subject may be any human who has cancer and has been diagnosed accordingly. The patient may be a child, an adolescent, an adult. The subject may or may not have been treated for symptoms associated with cancer. In an embodiment of the invention, the subject is or has been treated against cancer, for example by chemotherapy, radiotherapy, immunotherapy, hormonotherapy or any suitable methods. In another embodiment of the invention, the subject is not or has not yet been treated against cancer. The invention may optionally comprise determining one or more clinical factors of said subject, such as selected from sex, age, body mass index, health history.

A biological sample obtained from the patient can be any biological sample, such as tissue, blood, urine, whole cell lysate, biopsy, tumor, tumor cells. Methods of obtaining a biological sample from a patient are well known in the art and include obtaining samples from surgically excised tissue. Tissue, blood, urine, biopsy, tumor and cellular samples can also be obtained without the need for invasive surgery, for example by puncturing the subject with a fine needle and withdrawing cellular material or by biopsy. In certain embodiments, samples taken from a patient can be treated or processed to obtain processed biological samples such as supernatant, whole cell lysate, or fractions or extract from cells obtained directly from the patient. In other embodiments, biological samples issued from a patient can also be used with no further treatment or processing. In a preferred embodiment, the biological sample obtained from the subject is a tissue, in particular a tissue from a tumor or a tumor extract, preferably obtained by biopsy. A biological sample issued from a subject may, for example, be a sample removed or collected or susceptible of being removed or collected from an internal organ or tissue or tumor of said subject, in particular from tumor, or a biological fluid from said subject such as the blood, serum, plasma, tumor microenvironment or urine. A biological sample collected or removed from the subject may, for example, be a sample comprising cancer cells which have been or are susceptible of being removed or collected from a tissue, in particular a tumor, of said subject. A step for lysis of the cells, in particular lysis of the cancer cells contained in said biological sample, may be carried out in advance in order to render nucleic acids or, if appropriate, proteins and/or polypeptides and/or peptides, directly accessible to the analysis.

As used herein, “myeloid cells” refer to blood cells or tumor microenvironment cells that arise from a progenitor cell for granulocytes, monocytes, dendritic cells, erythrocytes, or platelet. Myeloid cells encompass macrophage cells, MDSCs, and myeloid dendritic cells, eosinophil cells, neutrophil cells, basophil cells, erythrocyte cells, and thrombocyte cells. In a particular embodiment, myeloid cells refer to Tumor Associated Macrophages, Monocytes, MDSCs, myeloid dendritic cells, and/or Tumor Associates Neutrophils.

### Anti-SIRPα Compounds

The composition to be used according to the present invention, or in the method according to the present invention, comprises an anti-SIRPα compound, in particular an anti-SIRPα antibody or an antigen-binding fragment thereof that inhibits the binding between SIRPα and CD47, preferably between human SIRPα and human CD47. The expression “anti-SIRPα compound” encompasses both anti-SIRPα antibody and antigen-binding fragment thereof that inhibits the binding between SIRPα and CD47, preferably between human SIRPα and human CD47.

In a particular embodiment, the anti-SIRPα antibody or antigen-binding fragment thereof is an antagonist of the binding between SIRPα and CD47; i.e. it reduces the interaction between SIRPα and CD47, preferably between human SIRPα and human CD47.

In a particular embodiment, the anti-SIRPα antibody or antigen-binding fragment thereof is an antagonist of signaling pathway induced by the interaction between SIRPα and CD47; i.e. it reduces or inhibits the intracellular molecular pathway normally activated in absence of the anti-SIRPα antibody or antigen-binding fragment thereof when SIRPα and CD47 interacts.

In a preferred embodiment, the anti-SIRPα antibody or antigen-binding fragment thereof does not inhibit the interaction between human SIRPg and human CD47.

In a particular embodiment, the anti-SIRPα antibody or antigen-binding fragment thereof binds specifically to human SIRPα v1 and to human SIRPα v2, allowing the treatment of patients expressing any combination of SIRPα alleles selected among SIRPα v1 and SIRPα v2.

In a particular embodiment, the anti-SIRPα antibody or antigen-binding fragment thereof does not specifically binds to human SIRPγ, in particular does not reduce or inhibit the interaction between human CD47 and human SIRPγ. In particular, the anti-SIRPα antibody or antigen-binding fragment thereof is not an antagonist of the human CD47/human SIRPγ interaction.

An antibody or an antigen-binding fragment thereof may be considered to inhibit the interaction between SIRPα and CD47 when the antibody or the antigen-binding fragment thereof has an antagonist effect on the CD47/SIRPα interaction. Used in the negative form, for example when the antibody, respectively the antigen-binding fragment thereof, does not inhibit the interaction between human SIRPg and human CD47, it means that the antibody, respectively the antigen-binding fragment thereof, does not have an antagonist effect on the CD47/SIRPg interaction. The decrease or the inhibition of the binding of (human) CD47 to (human) SIRPα means that the antibody or antigen-binding fragment thereof or antigen-binding antibody mimetic or modified antibody decreases the interaction between SIRPα and CD47, i.e. the antibody or antigen-binding fragment thereof partially or fully inhibits the binding of human CD47 to human SIRPα, or in other words, specifically binds to human SIRPα, and antagonizes the interaction between human SIRPα and human CD47. In particular, the anti-human SIRPα antibody or antigen-binding fragment thereof has the capability to decrease or inhibit the binding of (human) CD47 to (human) SIRPα by at least 50%, preferably 60%, more preferably 70%, more preferably 80% and most preferably 90%, and in a particular embodiment 100%, as compared to a negative control molecule, in a binding assay. In particular, the anti-SIRPα antibody or antigen-binding fragment thereof has the capability to reduce or inhibit the binding of human CD47 to human SIRPα from 50% to 100%, more preferably from 50% to 90%, as compared to a negative control molecule, in a binding assay.

In a particular embodiment of the invention, the anti-SIRPα antibody or antigen-binding fragment thereof is an anti-SIRPα antagonist antibody or antigen-binding fragment thereof, which antagonizes (i.e. reduces or inhibits) the binding between SIRPα and CD47, preferably between human SIRPα and human CD47.

In a particular embodiment of the invention, the anti-SIRPα antibody or antigen-binding fragment thereof comprises:
i) A heavy chain variable domain comprising the amino acid sequence set forth in SEQ ID No: 3, or in SEQ ID No: 4, or in SEQ ID No: 5; or in SEQ ID No: 6; or in SEQ ID No: 7; or in SEQ ID No: 8;
ii) A light chain variable domain comprising the amino acid sequence set forth in SEQ ID No: 9 or in SEQ ID No: 10.

In a particular embodiment of the invention, the anti-SIRPα antibody or antigen-binding fragment thereof comprises
(i) a heavy chain variable domain comprising:
   a heavy chain CDR1 (HCDR1) domain comprising or consisting of the amino acid sequence set forth in SEQ ID No. 15; and
   a heavy chain CDR2 (HCDR2) domain comprising or consisting of the amino acid sequence set forth in SEQ ID No. 16 or SEQ ID No. 17; and
   a heavy chain CDR3 (HCDR3) domain comprising or consisting of the amino acid sequence set forth in SEQ ID No. 18 or SEQ ID No. 19 or SEQ ID No. 20 or SEQ ID No. 21; and
(ii) a light chain variable domain comprising:
   a light chain CDR1 (LCDR1) domain comprising or consisting of the amino acid sequence set forth in SEQ ID No. 22; and
   a light chain CDR2 (LCDR2) domain comprising or consisting of the amino acid sequence set forth in SEQ ID No. 23; and
   a light chain CDR3 (LCDR3) domain comprising or consisting of the amino acid sequence set forth in SEQ ID No. 24.

In a particular embodiment, the anti-SIRPα antibody or antigen-binding fragment thereof comprises
a heavy chain variable domain comprising:
   a heavy chain CDR1 (HCDR1) domain comprising or consisting of the amino acid sequence set forth in SEQ ID No. 15; and
   a heavy chain CDR2 (HCDR2) domain comprising or consisting of the amino acid sequence set forth in SEQ ID No. 17; and
   a heavy chain CDR3 (HCDR3) domain comprising or consisting of the amino acid sequence set forth in SEQ ID No. 21; and
a light chain variable domain comprising:
   a light chain CDR1 (LCDR1) domain comprising or consisting of the amino acid sequence set forth in SEQ ID No. 22; and
   a light chain CDR2 (LCDR2) domain comprising or consisting of the amino acid sequence set forth in SEQ ID No. 23; and
   a light chain CDR3 (LCDR3) domain comprising or consisting of the amino acid sequence set forth in SEQ ID No. 24.

In a particular embodiment of the invention, the anti-SIRPα antibody or antigen-binding fragment thereof is a monoclonal antibody. In a particular embodiment of the invention, the anti-SIRPα antibody or antigen-binding fragment thereof is a humanized antibody. In a particular embodiment of the invention, the anti-SIRPα antibody or antigen-binding fragment thereof is a humanized monoclonal antibody. In a particular embodiment of the invention, the anti-SIRPα antibody or antigen-binding fragment thereof is selected from the group consisting of Fab, Fab′, Fab′-SH, Fv, single chain variable fragment (scFv), double chain variable fragment (dsFv) and (Fab′)2 fragments. In particular, said antibody or antigen-binding fragment thereof comprises a constant chain belonging to the subclass of IgG1, IgG2, IgG3 or IgG4, in particular the subclass of IgG1 or IgG4, and most particularly the subclass of IgG4.

In a particular embodiment of the invention, the anti-SIRPα antibody or antigen-binding fragment thereof comprises the heavy chain variable domain comprising the amino acid sequence set forth in SEQ ID No: 8 and the light chain variable domain comprising the amino acid sequence set forth in SEQ ID No: 10.

In a particular embodiment of the invention, the anti-SIRPα antibody comprises a heavy chain comprising the amino acid sequence set forth in SEQ ID No: 8 and a light chain variable domain comprising the amino acid sequence set forth in SEQ ID No: 10.

In a particular embodiment of the invention, anti-SIRPα antibody or antigen-binding fragment thereof comprises a heavy chain comprising the amino acid sequence set forth in SEQ ID No: 11, and a light chain comprising of the amino acid sequence set forth in SEQ ID No. 12.

In a particular embodiment of the invention, anti-SIRPα antibody comprises a heavy chain comprising the amino acid sequence set forth in SEQ ID No: 11, and a light chain comprising of the amino acid sequence set forth in SEQ ID No. 12.

In a particular embodiment of the invention, anti-SIRPα antibody or antigen-binding fragment thereof comprises a heavy chain comprising the amino acid sequence set forth in SEQ ID No: 13, and a light chain comprising of the amino acid sequence set forth in SEQ ID No. 12.

In a particular embodiment of the invention, anti-SIRPα antibody comprises a heavy chain comprising the amino acid sequence set forth in SEQ ID No: 13, and a light chain comprising of the amino acid sequence set forth in SEQ ID No. 12.

In a particular embodiment of the invention, the anti-SIRPα antibody or antigen-binding fragment thereof that may be used in the presently disclosed invention include SEQ ID NO: 8 and SEQ ID NO: 9 of WO 2020/099653, or a SIRPa-binding portion thereof, e.g., an antigen-binding fragment of an anti-SIRPa antibody, an anti-SIRPa antibody or antigen-fragment containing the CDRs of the foregoing, etc. which may optionally be linked to a pharmacokinetic enhancer.

Additional exemplary anti-SIRPa antibodies that may be used in the presently disclosed invention include SEQ ID NO: 7 and SEQ ID NO: 8 of WO 2019/023347, or SEQ ID NO: 15 and SEQ ID NO: 16 of WO 2019/023347, or a SIRPa-binding portion of any of the foregoing, e.g., an antigen-binding fragment of an anti-SIRPa antibody, an anti-SIRPa antibody or antigen-fragment containing the CDRs of any of the foregoing, etc. which may optionally be linked to a pharmacokinetic enhancer.

In a particular embodiment of the invention, the anti-SIRPα antibody or antigen-binding fragment thereof that may be used in the presently disclosed invention may be any one of the antibodies disclosed in the PCT publication published under reference WO2022/254379, in particular the antibodies referenced A, A4, A10, A11, E, E22, more particularly, the anti-SIRPα antibody or antigen-binding fragment thereof comprising the CDR disclosed in any one of tables 1 to 10 of WO2022/254379, most particularly the anti-SIRPα antibody or antigen-binding fragment thereof comprises the heavy chain variable region of SEQ ID No. 100 or SEQ ID No. 111 or SEQ ID No. 113 or SEQ ID No. 104 or SEQ ID No. 221 of WO2022/254379 and the light chain variable region of SEQ ID No. 105 or SEQ ID No. 125 or SEQ ID No. 126 or SEQ ID No. 109 or SEQ ID No. 222 of WO2022/254379, or has the heavy chain of SEQ ID No. 131 or SEQ ID No. 135 or SEQ ID No. 141 or SEQ ID No. 147 or SEQ ID No. 217 or SEQ ID No. 219 and the light chain of SEQ ID No. 174 or SEQ ID No. 178 or SEQ ID No. 184 or SEQ ID No. 190 or SEQ ID No. 218 or SEQ ID No. 220. In particular the anti-SIRPα antibody or antigen-binding fragment thereof that may be used in the presently disclosed invention may have any one of the following combinations of heavy chain variable region and light chain variable region: SEQ ID No. 100 and SEQ ID No. 105 or SEQ ID No. 104 and SEQ ID No. 109 or SEQ ID No. 113 and SEQ ID No. 125 or SEQ ID No. 111 and SEQ ID No. 126 or SEQ ID No. 221 and SEQ ID No. 222. In particular the anti-SIRPα antibody or antigen-binding fragment thereof that may be used in the presently disclosed invention may have any one of the following combinations of heavy chain and light chain: SEQ ID No. 131 and SEQ ID No. 174 or SEQ ID No. 135 and SEQ ID No. 178 or SEQ ID No. 141 and SEQ ID No. 184 or SEQ ID No. 147 and SEQ ID No. 190 or SEQ ID No. 217 and SEQ ID No. 218 or SEQ ID No. 219 and SEQ ID No. 220.

Additional exemplary anti-SIRPa antibodies that may be used in the presently disclosed invention include SEQ ID NO: 80 and SEQ ID NO: 67 of WO 2020/068752, SEQ ID NO: 85 and SEQ ID NO: 67 of WO 2020/068752, or SEQ ID NO: 138 and SEQ ID NO: 127 of WO 2020/068752, or a SIRPa-binding portion of any of the foregoing, e.g., an antigen-binding fragment of an anti-SIRPa antibody, an anti-SIRPa antibody or antigen-fragment containing the CDRs of any of the foregoing, etc. which may optionally be linked to a pharmacokinetic enhancer.

Additional exemplary anti-SIRPa antibodies that may be used in the presently disclosed invention include SEQ ID NO: 105 and SEQ ID NO: 124 of WO 2021/226576; SEQ ID NO: 108 and SEQ ID NO: 127 of WO 2021/226576; SEQ ID NO: 109 and SEQ ID NO: 128 of WO 2021/226576; SEQ ID NO: 119 and SEQ ID NO: 138 of WO 2021/226576; SEQ ID NO: 120 and SEQ ID NO: 139 of WO 2021/226576; SEQ ID NO: 121 and SEQ ID NO: 140 of WO 2021/226576; SEQ ID NO: 122 and SEQ ID NO: 141 of WO 2021/226576; wherein said antibody is optionally linked to a constant region (e.g., a human IgG1, IgG2, IgG3, or IgG4 constant region or variant thereof) a SIRPa-binding portion of any of the foregoing, e.g., an antigen-binding fragment of an anti-SIRPa antibody, an anti-SIRPa antibody or antigen-fragment containing the CDRs of any of the foregoing, etc. which may optionally be linked to a pharmacokinetic enhancer.

Additional exemplary anti-SIRPa antibodies that may be used in the presently disclosed invention include SEQ ID NO: 104 and SEQ ID NO: 123 of WO 2021/226591; SEQ ID NO: 106 and SEQ ID NO: 125 of WO 2021/226591; SEQ ID NO: 107 and SEQ ID NO: 126 of WO 2021/226591; SEQ ID NO: 110 and SEQ ID NO: 129 of WO 2021/226591; SEQ ID NO: 111 and SEQ ID NO: 130 of WO 2021/226591; SEQ ID NO: 112 and SEQ ID NO: 131 of WO 2021/226591; SEQ ID NO: 113 and SEQ ID NO: 132 of WO 2021/226591; SEQ ID NO: 114 and SEQ ID NO: 133 of WO 2021/226591; SEQ ID NO: 115 and SEQ ID NO: 134 of WO 2021/226591; SEQ ID NO: 116 and SEQ ID NO: 135 of WO 2021/226591; SEQ ID NO: 117 and SEQ ID NO: 136 of WO 2021/226591; SEQ ID NO: 118 and SEQ ID NO: 137; wherein said antibody is optionally linked to a constant region (e.g., a human IgG1, IgG2, IgG3, or IgG4 constant region or variant thereof) a SIRPa-binding portion of any of the foregoing, e.g., an antigen-binding fragment of an anti-SIRPa antibody, an anti-SIRPa antibody or antigen-fragment containing the CDRs of any of the foregoing, etc. which may optionally be linked to a pharmacokinetic enhancer.

Additional exemplary anti-SIRPa antibodies that may be used in the presently disclosed invention include the VH region set forth in SEQ ID NO: 169, and the VL region set forth in SEQ ID NO: 170 of WO2021174127; the VH region set forth in SEQ ID NO: 171, and the VL region set forth in SEQ ID NO: 172 of WO2021174127; the VH region set forth in SEQ ID NO: 173, and the VL region set forth in SEQ ID NO: 174 of WO2021174127; the VH region set forth in SEQ ID NO: 175, and the VL region set forth in SEQ ID NO: 176 of WO2021174127; the VH region set forth in SEQ ID NO: 177, and the VL region set forth in SEQ ID NO: 178 of WO2021174127; the VH region set forth in SEQ ID NO: 179, and the VL region set forth in SEQ ID NO: 180 of WO2021174127; the VH region set forth in SEQ ID NO: 181, and the VL region set forth in SEQ ID NO: 182 of WO2021174127; the VH region set forth in SEQ ID NO: 183, and the VL region set forth in SEQ ID NO: 184 or 227 of WO2021174127; the VH region set forth in SEQ ID NO: 185, and the VL region set forth in SEQ ID NO: 186 of WO2021174127; the VH region set forth in SEQ ID NO: 187, and the VL region set forth in SEQ ID NO: 188 of WO2021174127; the VH region set forth in SEQ ID NO: 189, and the VL region set forth in SEQ ID NO: 190 of WO2021174127; the VH region set forth in SEQ ID NO: 191, and the VL region set forth in SEQ ID NO: 192 of WO2021174127; the VH region set forth in SEQ ID NO: 193, and the VL region set forth in SEQ ID NO: 194 of WO2021174127; the VH region set forth in SEQ ID NO: 195, and the VL region set forth in SEQ ID NO: 196 of WO2021174127; the VH region set forth in SEQ ID NO: 197, and the VL region set forth in SEQ ID NO: 198 of WO2021174127; the VH region set forth in SEQ ID NO: 199, and the VL region set forth in SEQ ID NO: 200 of WO2021174127; the VH region set forth in SEQ ID NO: 201, and the VL region set forth in SEQ ID NO: 202 of WO2021174127; the VH region set forth in SEQ ID NO: 203, and the VL region set forth in SEQ ID NO: 204 of WO2021174127; the VH region set forth in SEQ ID NO: 205, and the VL region set forth in SEQ ID NO: 206 of WO2021174127; the VH region set forth in SEQ ID NO: 207, and the VL region set forth in SEQ ID NO: 208 of WO2021174127; the VH region set forth in SEQ ID NO: 209, and the VL region set forth in SEQ ID NO: 210 of WO2021174127; the VH region set forth in SEQ ID NO: 211, and the VL region set forth in SEQ ID NO: 212 of WO2021174127; the VH region set forth in SEQ ID NO: 213, and the VL region set forth in SEQ ID NO: 214 of WO2021174127; the VH region set forth in SEQ ID NO: 215, and the VL region set forth in SEQ ID NO: 216 of WO2021174127; the VH region set forth in SEQ ID NO: 217, and the VL region set forth in SEQ ID NO: 218 of WO2021174127; the VH region set forth in SEQ ID NO: 219, and the VL region set forth in SEQ ID NO: 220 of WO2021174127; the VH region set forth in SEQ ID NO: 221, and the VL region set forth in SEQ ID NO: 222 of WO2021174127; or the VH region set forth in SEQ ID NO 223, and the VL region set forth in SEQ ID NO: 224 of WO2021174127; wherein said antibody is optionally linked to a constant region (e.g., a human IgG1, IgG2, IgG3, or IgG4 constant region or variant thereof) a SIRPa-binding portion of any of the foregoing, e.g., an antigen-binding fragment of an anti-SIRPa antibody, an anti-SIRPa antibody or antigen-fragment containing the CDRs of any of the foregoing, etc. which may optionally be linked to a pharmacokinetic enhancer.

Additional exemplary anti-SIRPa antibodies that may be used in the presently disclosed invention include SEQ ID NO: 35 and SEQ ID NO: 41 of WO 2019/226973, or a SIRPa-binding portion thereof, e.g., an antigen-binding fragment of an anti-SIRPa antibody, an anti-SIRPa antibody or antigen-fragment containing the CDRs of the foregoing, etc. which may optionally be linked to a pharmacokinetic enhancer.

Additional exemplary anti-SIRPa antibodies that may be used in the presently disclosed invention include SEQ ID NO: 104 and SEQ ID NO: 102 of WO 2018/190719; or SEQ ID NO: 1 and SEQ ID NO: 2 of WO 2018/190719 or a SIRPa-binding portion of any of the foregoing, e.g., an antigen-binding fragment of an anti-SIRPa antibody, an anti-SIRPa antibody or antigen-fragment containing the CDRs of any of the foregoing, etc. which may optionally be linked to a pharmacokinetic enhancer.

Additional exemplary anti-SIRPa antibodies that may be used in the presently disclosed invention include SEQ ID NO: 64 and SEQ ID NO: 78 of US20210347908; SEQ ID NO: 65 and SEQ ID NO: 79 of US20210347908; SEQ ID NO: 65 and SEQ ID NO: 80 of US20210347908; SEQ ID NO: 66 and SEQ ID NO: 81 of US20210347908; SEQ ID NO: 65 and SEQ ID NO: 82 of US20210347908; SEQ ID NO: 67 and SEQ ID NO: 83 of US20210347908; SEQ ID NO: 68 and SEQ ID NO: 82 of US20210347908; or SEQ ID NO: 65 and SEQ ID NO: 84 of US20210347908; wherein said antibody is optionally linked to a constant region (e.g., a human IgG1, IgG2, IgG3, or IgG4 constant region or variant thereof) a SIRPa-binding portion of any of the foregoing, e.g., an antigen-binding fragment of an anti-SIRPa antibody, an anti-SIRPa antibody or antigen-fragment containing the CDRs of any of the foregoing, etc. which may optionally be linked to a pharmacokinetic enhancer.

Additional exemplary anti-SIRPa antibodies that may be used in the presently disclosed invention include SEQ ID NO: 81 and SEQ ID NO: 64 of WO 2020/102422; SEQ ID NO: 82 and SEQ ID NO: 65 of WO 2020/102422; SEQ ID NO: 83 and SEQ ID NO: 66 of WO 2020/102422; SEQ ID NO: 84 and SEQ ID NO: 67 of WO 2020/102422; SEQ ID NO: 85 and SEQ ID NO: 68 of WO 2020/102422; SEQ ID NO: 86 and SEQ ID NO: 69 of WO 2020/102422; SEQ ID NO: 87 and SEQ ID NO: 70 of WO 2020/102422; SEQ ID NO: 88 and SEQ ID NO: 71 of WO 2020/102422; SEQ ID NO: 89 and SEQ ID NO: 72 of WO 2020/102422; SEQ ID NO: 90 and SEQ ID NO: 73 of WO 2020/102422; SEQ ID NO: 91 and SEQ ID NO: 74 of WO 2020/102422; SEQ ID NO: 91 and SEQ ID NO: 75 of WO 2020/102422; SEQ ID NO: 91 and SEQ ID NO: 76 of WO 2020/102422; SEQ ID NO: 92 and SEQ ID NO: 74 of WO 2020/102422; SEQ ID NO: 92 and SEQ ID NO: 75 of WO 2020/102422; SEQ ID NO: 92 and SEQ ID NO: 76 of WO 2020/102422; SEQ ID NO: 93 and SEQ ID NO: 74 of WO 2020/102422; SEQ ID NO: 93 and SEQ ID NO: 75 of WO 2020/102422; SEQ ID NO: 93 and SEQ ID NO: 76 of WO 2020/102422; SEQ ID NO: 94 and SEQ ID NO: 74 of WO 2020/102422; SEQ ID NO: 94 and SEQ ID NO: 75 of WO 2020/102422; SEQ ID NO: 94 and SEQ ID NO: 76 of WO 2020/102422; SEQ ID NO: 84 and SEQ ID NO: 77 of WO 2020/102422; SEQ ID NO: 95 and SEQ ID NO: 78 of WO 2020/102422; SEQ ID NO: 95 and SEQ ID NO: 79 of WO 2020/102422; SEQ ID NO: 95 and SEQ ID NO: 80 of WO 2020/102422; SEQ ID NO: 96 and SEQ ID NO: 78 of WO 2020/102422; SEQ ID NO: 96 and SEQ ID NO: 79 of WO 2020/102422; SEQ ID NO: 96 and SEQ ID NO: 80 of WO 2020/102422; SEQ ID NO: 97 and SEQ ID NO: 78 of WO 2020/102422; SEQ ID NO: 97 and SEQ ID NO: 79 of WO 2020/102422; SEQ ID NO: 97 and SEQ ID NO: 80 of WO 2020/102422; or SEQ ID NO: 89 and SEQ ID NO: 72; wherein said antibody is optionally linked to a constant region (e.g., a human IgG1, IgG2, IgG3, or IgG4 constant region or variant thereof) a SIRPa-binding portion of any of the foregoing, e.g., an antigen-binding fragment of an anti-SIRPa antibody, an anti-SIRPa antibody or antigen-fragment containing the CDRs of any of the foregoing, etc. which may optionally be linked to a pharmacokinetic enhancer.

Additional exemplary anti-SIRPa antibodies that may be used in the presently disclosed invention include BR105 (Bioray Biopharmaceutical Co. Ltd.; see clinical trial no. NCT05351697); ELA026 (Electra Therapeutics, Inc.; see clinical trial no. NCT05416307); IBI397 (Innovent Biologics (Suzhou) Co. Ltd.; Alector; see clinical trial no. NCT05245916); BSI-050 (Biosion); BSI-082 (Biosion); ES004 (ELPIscience); APX701 (Apexigen); and BYON4228 (Byondis).

Anti-SIRPa antibodies of the present invention may include an anti-SIRPa antibody according to any one of WO0066159, WO0140307, WO200140307, WO2009131453, WO2013056352, WO2014149477, WO2014186761, WO2015138600, WO2016063233, WO2016205042, WO2017178653, WO2018008470, WO2018026600, WO2018057669, WO2018107058, WO2018141964, WO2018160739, WO2018190719, WO2018210793, WO2019023347, WO2019183266, WO2019200462, WO2019226973, WO2020006374, WO2020013170, WO2020033646, WO2020068752, WO2020102422, WO2020099653, WO2020180811, WO2020247820, WO2021022044, WO2021032078, WO2021076908, WO2021129697, WO2021174127, WO2021185273, CN111635458, WO2021222746, WO2021226576, WO2021226591, CN113735973, CN111995682, CN112010979, or CN112574310, each of which is hereby incorporated by reference in its entirety, or a SIRPa-binding portion of any of the foregoing, e.g., an anti-SIRPa antibody or CD47 fragment contained in any of the foregoing, a SIRPa-binding fragment thereof (e.g., antigen-binding fragment of an anti-SIRPa antibody), an anti-SIRPa antibody or antigen-fragment containing the CDRs thereof, etc. which may optionally be linked to a pharmacokinetic enhancer.

All of these references disclosing anti-SIRPα antibodies or antigen-binding fragments thereof are incorporated by reference herein. Any one of these antibodies can be used according to any method or use disclosed in the present invention, in the same manner as the antibody used in the working examples of the present disclosure.

### Patients Likely to Positively Respond to a Treatment with the Anti-SIRPα Compounds as Defined Herein

In an aspect of the invention, it is provided a composition comprising an anti-SIRPα antibody or an antigen-binding fragment thereof that inhibits the binding between SIRPα and CD47, preferably between human SIRPα and human CD47, for use in the treatment of a cancer in a patient, the patient having:
at least 55%, in particular at least 60% in particular at least 65.3% of its myeloid cells present within the microenvironment of a tumor, in particular Tumor Associated Macrophages, Monocytes, myeloid dendritic cells, MDSCs, and/or Tumor Associates Neutrophils, that express the biological markers CD11b and SIRPα

In a preferred embodiment, a patient has at least 55%, or at least 60%, or at least 61%, or at least 62% or at least 63%, or at least 64% or at least 65%, in particular at least 65.3%, or at least 70%, or at least 75%, or at least 80%, or at least 85%, or at least 90%, of its myeloid cells present within the microenvironment of a tumor, in particular Tumor Associated Macrophages, Monocytes, myeloid dendritic cells, MDSCs, and/or Tumor Associates Neutrophils, that express the biological markers CD11b and SIRPα. In a particular embodiment, a patient has at least 55%, in particular, or at least 60%, or at least 61%, or at least 62% or at least 63%, or at least 64% or at least 65%, in particular at least 65.3%, or at least 70%, or at least 75%, or at least 80%, or at least 95%, or at least 90%, of its Tumor Associated Macrophages, Monocytes, myeloid dendritic cells, Tumor Associates Neutrophils and/or MDSCs, in particular MDSCs, within the microenvironment of a tumor that express the biological markers CD11b and SIRPα. When several types of myeloid cells are selected for the measure of the percentage of myeloid cells that express CD11b and SIRPα, the percentage of cells that express CD11b and SIRPα expressed is measured in each type of myeloid cells that has been selected, and the measures are pooled for obtaining the measure of the expression of CD11b and SIRPα in the myeloid cells.

Detection of myeloid cells, including Tumor Associated Macrophages, Monocytes, myeloid dendritic cells, Tumor Associates Neutrophils and/or MDSCs, expressing these markers can be performed according to methods known in the art from a biological sample previously obtained from the patient, like an immunoassay, a gene expression profile, a fluorescence detection, an enzymatic activity a assay, chemiluminescence detection, immunohistochemistry, polymerase chain reaction, reverse-transcriptase-polymerase chain reaction, antibody binding, receptor binding arrays, target specific primers extension, ELISA, radioactive labelling, of SIRPα and CD11b. As an example, it is possible to measure the expression of CD11b and SIRPα on the surface of myeloid cells present in a biological sample by immunohistochemistry or by flow cytometry using several, different antibodies or fluorochromes known to interact with one of the listed markers.

In a preferred embodiment, a patient has at least 55%, in particular at least 60% in particular at least 65.3 of its myeloid cells present within the microenvironment of a tumor, in particular Tumor Associated Macrophages, Monocytes, MDSCs, myeloid dendritic cells, and/or Tumor Associates Neutrophils, that express the biological markers CD11b and SIRPα.

In an embodiment of the invention, the anti-SIRPα antibody or an antigen-binding fragment thereof that inhibits the binding between SIRPα and CD47 is administered at least once to a patient whose at least 55%, in particular at least 60% in particular at least 65.3% of its myeloid cells, in particular Tumor Associated Macrophages, Monocytes, myeloid dendritic cells, Tumor Associates Neutrophils and/or MDSCs, more particularly MDSCs, present within the microenvironment of a tumor express SIRPα and CD11b.

In a particular embodiment of the invention, it is provided a composition comprising an anti-SIRPα antibody or an antigen-binding fragment thereof that inhibits the binding between SIRPα and CD47, in particular between human SIRPα and human CD47, for use in the treatment of a patient who has a cancer, the patient having:
at least 55%, in particular at least 60% in particular at least 65.3%, most preferably at least 70%, of its myeloid cells, present within the microenvironment of a tumor, in particular Tumor Associated Macrophages, Monocytes, myeloid dendritic cells, and/or Tumor Associates Neutrophils, that express the biological markers CD11b and SIRPα.

In a preferred embodiment, the patient has myeloid cells within the microenvironment of the tumor. These cells can be detected in a biological sample, in particular in a biopsy, obtained from the patient. Cell detection can be performed by method known in the art, for example par detecting specifical molecular markers associated with myeloid cells in IHC or flow cytometry assay.

In a preferred embodiment, the composition is for use in the treatment of a patient having a SIRPα-positive cancer, a PD-1-positive cancer or a PD-L1-positive cancer, in particular a cancer with solid tumors expressing or over-expressing SIRPα, PD-1 and/or PD-L1, wherein the patient is identified for exhibiting the biomarkers disclosed according to the invention.

In a preferred embodiment, the composition is for use in the treatment of a patient having a solid cancer, in particular a cancer with advanced solid tumors, more particularly an adrenal cancer, a biliary tree cancer, a breast cancer, a cervical cancer, a colorectal cancer, an endometrial cancer, a gastrointestinal cancer, a head and neck cancer, a kidney cancer, a liver cancer, a lung cancer, a melanoma, a non-small cells lung cancer, an ovarian cancer, a hepatocellular carcinoma, a pancreas cancer, a parotid cancer, a prostate cancer or an uterine cancer, more particularly an ovarian cancer, a breast cancer in particular a Triple Negative Breast cancer, lung cancer more particularly Non-Small Cells Lung Cancer (NSCLC), cervical cancer, and colorectal cancer, most particularly Non-Small Cell Lung Cancer (NSCLC), cervical cancer, or colorectal cancer, wherein the patient is identified for exhibiting the biomarkers disclosed according to the invention.

### Second Therapeutic Compound that can be Administered to the Patients

In some embodiments, the anti-SIRPα compound for use in the method of treatment of the invention or for use according to the invention is administered to the patient in combination with a standard or conventional or standard of care treatment. The present invention relates thus to the combination of an anti-SIRPα compound for use in the method of treatment of the invention or for use according to the invention with a conventional treatment for use in the treatment of cancer. As used herein, the term “standard or conventional treatment or standard of care treatment” refers to any treatment of cancer (drug, surgery, radiotherapy, etc.) usually administered to a patient who suffers from cancer.

In some embodiments, the anti-SIRPα compound for use in the method of treatment of the invention or for use according to the invention is administered to the patient in a combination treatment with at least one further therapeutic agent suitable for treating cancers. Such administration may be simultaneous, separate or sequential. For simultaneous administration, the agents may be administered as one composition or as separate compositions, as appropriate. The further therapeutic agent is typically relevant for the disorder to be treated. Exemplary therapeutic agents include other anti-cancer antibodies, cytotoxic agents, chemotherapeutic agents, anti-angiogenic agents, anti-cancer immunogens, cell cycle control/apoptosis regulating agents, hormonal regulating agents, and other agents described below.

The anti-SIRPα antibody or antigen-binding fragment thereof may be administered in a combination treatment with at least one other therapeutic compound. The anti-SIRPα antibody or antigen-binding fragment thereof may also be administered in a combination treatment with several (i.e. more than one, like two, three of four for example) other therapeutic compounds.

In some embodiments, the anti-SIRPα compound for use in the method of treatment of the invention or for use according to the invention is used in a combination treatment with a second therapeutic agent.

The second therapeutic agent may be selected from the group consisting of chemotherapeutic agents, radiotherapy agents, immunotherapeutic agents, hormonotherapeutic agents, cell therapy agents, antibiotics and probiotics, in particular immunotherapeutic agents selected from the group consisting of immune checkpoint inhibitors or activators of adaptive immune cells, particularly selected from the group consisting of anti-PD-L1, anti-PD-1, anti_LAG-3, anti-CTLA4, anti-CD137, anti-CD2, anti-CD28, anti-CD40, anti-HVEM, anti-BTLA, anti-CD160, anti-TIGIT, anti-TIM-1/3, anti-LAG-3, anti-2B4, anti-VISTA, anti-OX40, anti-CD40 agonist, CD40-L, TLR agonists, anti-ICOS, ICOS-L, STING agonist, IDO inhibitor, oncolytic virus agonists, and B-cell receptor agonists, in particular wherein such therapeutic agent is an antibody. The combination of agents is for use in the treatment of cancer.

In one embodiment, the anti-PD1 antibody can be selected from the group consisting of Pembrolizumab (also known as Keytruda lambrolizumab, MK-3475), Nivolumab (Opdivo, MDX-1106, BMS-936558, ONO-4538), Pidilizumab (CT-011), Cemiplimab (Libtayo), Ezabenlimab (a humanized programmed cell death 1 (PD-1)-targeting monoclonal antibody), Camrelizumab, AUNP12, AMP-224, AGEN-2034, BGB-A317 (Tisleizumab), PDR001 (spartalizumab), MK-3477, SCH-900475, PF-06801591, JNJ-63723283, genolimzumab (CBT-501), LZM-009, BCD-100, SHR-1201, BAT-1306, AK-103 (HX-008), MEDI-0680 (also known as AMP-514) MEDI0608, JS001 (see Si-Yang Liu et al., J. Hematol. Oncol. 10:136 (2017)), BI-754091, CBT-501, INCSHR1210 (also known as SHR-1210), TSR-042 (also known as ANB011), GLS-010 (also known as WBP3055), AM-0001 (Armo), STI-1110 (see WO 2014/194302), AGEN2034 (see WO 2017/040790), MGA012 (see WO 2017/19846), or IBI308 (see WO 2017/024465, WO 2017/025016, WO 2017/132825, and WO 2017/133540), monoclonal antibodies 5C4, 17D8, 2D3, 4H1, 4A11, 7D3, and 5F4, described in WO 2006/121168.

In a particular embodiment of the invention, the second therapeutic agent is an anti-PD-1 antagonist or an anti-PD-L1 antagonist. In a particular embodiment of the invention, the second therapeutic agent is an anti-PD-1 antibody or an anti-PD-L1 antibody, more particularly an anti-PD-1 antagonist antibody or an anti-PD-L1 antagonist antibody. In a particular embodiment of the invention, the second therapeutic agent is a PD-1 antagonist; and is in particular selected from the group consisting of the following antibodies: pembrolizumab; nivolumab; pidilizumab; tislelizumab; spartalizumab; ezabenlimab; preferably ezabenlimab. In a particular embodiment of the invention, the second therapeutic agent is an anti-PD-L1 antagonist; and is in particular selected from the group consisting of Avelumab (Bavencio), durvalumab (Imfinzi), atezolizumab (Tecentriq).

In a particular embodiment of the invention, the second therapeutic agent is administered to the patient in any therapeutically effective amount. In certain embodiments, the therapeutically acceptable amount is between about 0.1 mg and about 50 mg per kg of weight of the patient (agent/body weight of the patient).

In a particular embodiment, the second therapeutic agent is selected among the group consisting of immune checkpoint inhibitors or activators, more particularly the second therapeutic agent is an anti-PD-1 compound or an anti-PD-L1 compound. In a preferred embodiment, the second therapeutic agent is a PD-1 antagonist compound, anti-PD1-antagonist antibody. In a preferred embodiment, the second therapeutic agent is an anti-PD-L1 antagonist compound, more particularly an anti-PDL1-antagonist antibody, in particular avelumab (Bavencio), durvalumab (Imfinzi), atezolizumab (Tecentriq).

The second therapeutic agent may be administered concurrently or separately, in particular subsequently to or sequentially, with the anti-SIRPα antibody or antigen-binding fragment thereof. The second therapeutic agent may be administered according to the same dosing cycle as the anti-SIRPα antibody or antigen-binding fragment thereof, either at the same time, or separately in time. The invention also concerns the anti-SIRPα compound according to any embodiment disclosed herein, either alone or in combination with a second therapeutic agent, and/or with a pharmaceutical suitable vehicle as defined here in, for use in a combination therapy for treating cancer, with another treatment including the use of a medicament comprising a chemotherapeutic agent, a radiotherapy agent, an immunotherapeutic agent (such as a tumor-targeting monoclonal antibody), an hormonotherapeutic agent, a cell therapy agents (such as CAR-T cells), an immunosuppressive agent, a pro-apoptotic agent, an antibiotic, a targeted cancer therapy, and/or a probiotic, in particular for simultaneous, separated, or sequential administration to a patient in need thereof.

The invention also concerns the use of the anti-SIRPα compound according to any embodiment disclosed herein, either alone or in combination with a second therapeutic agent, and/or with a pharmaceutical suitable vehicle as defined here in, for use in a combination therapy for treating cancer, with another treatment including surgery, chemotherapy, radiation therapy, stem cell therapy, immunotherapy, targeted therapy, in particular for simultaneous, separated, or sequential administration to a patient in need thereof and under one of the listed therapies.

### Administration Route

The used and methods described herein permit the administration of the anti-SIRPα compound via any acceptable route. In typical embodiments, the pharmaceutical composition is formulated in accordance with routine procedures as a pharmaceutical composition adapted for intravenous administration to human beings. Typically, compositions for intravenous administration are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration. In some embodiments, the anti-SIRPα antibody or antigen-binding fragment thereof is administered orally, sub-cutaneously, parenterally (e.g. in the form of a liquid), rectally (i.e. in the form of a suppository), topically (e.g. in the form of a transdermal patch, ointment, or cream), or intranasally. In other words, the uses of the anti-SIRPα compound and the methods of using such a compound may be performed through an administration route encompassing oral administration, local administration to the gastrointestinal (GI) tract, in particular through oral administration, in particular for the treatment of cancers related to the digestive tract. In particular, provided herein are compositions suitable for systemic administration, in particular for parenteral or enteral administration, in particular for intravenous injection or infusion or oral administration. Enteric administration may be either a local administration to the digestive tract, or a systemic administration. The route of administration may encompass the use of a device allowing administration in particular injection or infusion of said composition (a “delivery device”). Examples of administration routes may include but are not limited to the use of the active compounds as a solution, in particular a sterile aqueous solution, suspension, as a solid, in particular a freeze-dried or a lyophilized solid, adsorbed on a patch, suspended or reconstituted and administrated as a solution, as a pill, tablet or other solid form suitable for oral administration, in particular with delayed or extended release. Preferably, the anti-SIRPα compound is administered sub-cutaneously or intravenously, preferentially intravenously.

In an embodiment of the invention, it is provided a kit comprising the anti-SIRPα compound (i.e. the antibody or antigen-binding fragment thereof) for use according to any one of the embodiments disclosed herein, and a device suitable for a local administration, in particular a subcutaneous or oral delivery device, in particular a device comprising a pre-filled syringe, or in particular a needle-free device. In particular, the device suitable for local administration comprised the anti-SIRPα compound at the prescribed dose for direct administration of the anti-SIRPα compound to the patient, without the need to dilute, to complete or to reconstitute the product before administration. Optionally associated with such kit(s) can be a notice in the form prescribed by a governmental agency regulating the manufacture, use or sale of pharmaceuticals or biological products, which notice reflects approval by the agency of manufacture, use or sale for human administration.

### Cancers

The uses described herein as well as the methods described herein may be useful in the treatment of cancer. In particular, the uses described herein as well as the methods described herein may be useful in the treatment of solid cancers and liquid cancers. The terms “cancer” has its general meaning in the art and refers to a group of diseases involving abnormal cell growth with the potential to invade or spread to other parts of the body. The term “cancer” encompasses both primary and metastatic cancers. Examples of cancers that may treated by methods and compositions of the invention include, but are not limited to, cancer from the bladder, blood, bone, bone marrow, brain, breast cancer, including Triple-Negative Breast cancer, colon, oesophagus, gastrointestinal, gum, head, kidney, liver, lung, nasopharynx, neck, ovary, prostate, skin, stomach, testis, tongue, or uterus. In a particular aspect of the invention, the uses and methods described herein are for the treatment of a cancer in a patient wherein the cancer is one of ovarian cancer, pancreas cancer, Vater's ampulla cancer, Micro Satellite Stable (MSS) cancer, Micro Satellite Instable (MSI) cancer, colorectal cancer, in particular MSI and MSS colorectal cancers, fibrolamellar cancer, breast cancer, melanoma, kidney cancer, lung cancer, in particular non-small cell lung cancer (NSCLC), head and neck cancer, in particular head and neck squamous cell carcinoma (HNSCC), gastric cancer, liver cancer, endometrial cancer, and hepatocellular carcinoma cancer.

The uses described herein as well as the methods described herein may be useful in the treatment of cancers wherein cancer cells express antigens with low tumor specificity; like CD47 and PD-L1 antigen(s).

In a particular aspect, the uses described herein as well as the methods described herein may be useful in the treatment of SIRPα-, CD47-, PD-L1- or PD-1-positive cancers (i.e. cancers wherein tumor cells or immune cells express SIRPα-, CD47-, PD-L1- and/or PD-1). The treated patient may have been diagnosed with a SIRPα-positive cancer, a CD47-positive cancer, a PD-1-positive cancer or a PD-L1-positive cancer, in particular a cancer with solid tumors expressing or over-expressing SIRPα, CD47, PD-1 and/or PD-L1. A SIRPα-, CD47-, PD-L1- or PD-1-positive cancer is a cancer wherein tumor cells or immune cells express SIRPα, CD47, PD-L1 and/or PD-1. “SIRPα, CD47-, -PD-L1- or PD-1-positive tumor cell” designates a tumor cell or immune cell expressing SIRPα, CD47, PD-L1 or PD-1 at their cell surface. A cancer may be classified in the subset of SIRPα, CD47, PD-L1- or PD-1-positive cancer by ImmunoHistoChemistry using a monoclonal antibody directed against SIRPα, CD47, PD-1 or PD-L1.

In a particular aspect of the invention, the uses and methods described herein are for the treatment of a cancer in a patient who has been diagnosed with a cancer with solid tumors, in particular with advanced solid tumors, more particularly an adrenal cancer, a biliary tree cancer, a breast cancer, a cervical cancer, a colorectal cancer, an endometrial cancer, a gastrointestinal cancer, a head and neck cancer, a kidney cancer, a liver cancer, a lung cancer, a melanoma, a non-small cells lung cancer, an ovarian cancer, a hepatocellular carcinoma, a pancreas cancer, a parotid cancer, a prostate cancer or an uterine cancer, more particularly an ovarian cancer, a breast cancer in particular a Triple Negative Breast cancer, lung cancer more particularly Non-Small Cells Lung Cancer (NSCLC), cervical cancer, and colorectal cancer most particularly Non-Small Cell Lung Cancer (NSCLC), cervical cancer, or colorectal cancer. A cancer with solid tumor may not contain any liquid or cysts. Solid tumors may correspond to either sarcomas or carcinomas.

In a particular aspect of the invention, the uses and methods described herein are for the treatment of a cancer in a patient who has a liquid cancer or a solid cancer, in particular a cancer from the bladder, blood, bone, bone marrow, brain, breast cancer, including Triple-Negative Breast cancer, colon, oesophagus, gastrointestinal, gum, head, kidney, liver, lung, nasopharynx, neck, ovary, prostate, skin, stomach, testis, tongue, or uterus. In a particular aspect of the invention, the uses and methods described herein are for the treatment of a cancer in a patient wherein the cancer is one of ovarian cancer, pancreas cancer, Vater's ampulla cancer, Micro Satellite Stable (MSS) cancer, Micro Satellite Instable (MSI) cancer, colorectal cancer, in particular MSI and MSS colorectal cancers, fibrolamellar cancer, breast cancer, melanoma, kidney cancer, lung cancer, in particular non-small cell lung cancer (NSCLC), head and neck cancer, in particular head and neck squamous cell carcinoma (HNSCC), gastric cancer, liver cancer, endometrial cancer, and hepatocellular carcinoma cancer, the cancer being positive to SIRPα, CD47, PD-L1 or PD1 as detailed here above.

### Patients

In a particular aspect of the invention, the uses and methods described herein are for the treatment of a patient who has been, prior to the use, treated for their cancer and has shown resistance to the treatment and/or disease progression despite being treated. The prior treatment may encompass any standard of conventional treatment of cancer. The term “standard or conventional treatment” refers to any treatment of cancer (drug, surgery, radiotherapy, etc.) usually administrated to a patient who suffers from cancer.

In a particular aspect of the invention, the uses and methods described herein are for the treatment of a patient who has been treated, is treated or will be treated with an immune checkpoint inhibitor.

In a particular aspect of the invention, the uses and methods described herein are for the treatment of a patient who has been treated with an immune checkpoint inhibitors or activators, in particular with an anti-PD-L1, anti-PD-1, anti_LAG-3, anti-CTLA4, anti-CD137, anti-CD2, anti-CD28, anti-CD40, anti-HVEM, anti-BTLA, anti-CD160, anti-TIGIT, anti-TIM-1/3, anti-LAG-3, anti-2B4, anti-VISTA, anti-OX40, anti-CD40 agonist, CD40-L, TLR agonists, anti-ICOS, ICOS-L, STING agonist, IDO inhibitor, oncolytic virus agonists, and B-cell receptor agonists, more particularly with an anti-PD-1 or an anti-PD-L1 antibody, and did not positively respond to the administration of the immune checkpoint inhibitors or activators (i.e. the patients show disease progression and/or do not show disease regression).

Accordingly, the uses and methods described herein may be for monotherapy or combination therapy for the treatment of a patient as identified above.

In a particular aspect of the invention, the uses and methods described herein are for the treatment of a patient who has not been treated, is not treated or will not be treated with an immune checkpoint inhibitor.

In a particular aspect of the invention, the uses and methods described herein are for the treatment of a patient who has not been treated with an anti-PD-1 antibody or anti-PD-L1 antibody, in particular an anti-PD-1 antagonist antibody or anti-PD-L1 antagonist antibody prior to the administration of the anti-SIRPα compound.

In a particular embodiment, the patient has at least one SIRPα V1 allele (i.e. is either homozygous and has two SIRPα V1 alleles or is heterozygous for SIRPα and has one SIRPα V1 allele). In a particular embodiment, the patient is homozygous for SIRPα and is SIRPα V1/SIRPα V1. In a particular embodiment, the patient is heterozygous for SIRPα and is SIRPα V1/SIRPα V2.

In a particular embodiment, the patient has at least one SIRPα V2 allele (i.e. is either homozygous and has two SIRPα V2 alleles or is heterozygous for SIRPα and has one SIRPα V2 allele). In a particular embodiment, the patient is homozygous for SIRPα and is SIRPα V2/SIRPα V2.

In a particular other aspect of the invention, the uses and methods described herein are performed in a combination therapy to sensitize the patient to a treatment with a second therapeutic agent, in particular with an immune checkpoint inhibitor or activator, more particularly with an anti-PD-1 antagonist antibody or with an anti-PD-L1 antibody, the anti-SIRPα antibody or antigen-binding fragment thereof being administered during a first period or cycle, and the second therapeutic agent, in particular an immune checkpoint inhibitor or activator, more particularly an anti-PD-1 antagonist antibody or with an anti-PD-L1 antibody, being administered during a second, subsequent, period of time or cycle, wherein second therapeutic agent, in particular the immune checkpoint inhibitor or activator, more particularly the anti-PD-1 antagonist antibody or the anti-PD-L1 antibody is not administered during the first period of time or cycle.

### Methods of Diagnostic and Treatment

In an embodiment, the invention concerns a method for determining whether a therapy for the treatment of a cancer wherein such therapy encompasses anti-SIRPα antibody or an antigen-binding fragment thereof that inhibits the binding between SIRPα and CD47, in particular between human SIRPα and human CD47, is likely to be effective in a patient having a cancer, the method comprising:
Providing a biological sample, in particular a blood sample, a plasma sample, a serum sample, a biopsy and/or a tumor sample, previously obtained from the patient,
Determining or measuring the presence of myeloid cells, in particular Tumor Associated Macrophages, Monocytes, myeloid dendritic cells, MDSCs, and/or Tumor Associates Neutrophils, expressing CD11b and SIRPα in the biological sample,
if myeloid cells expressing CD11b and SIRPα biomarkers are present in the biological sample, the patient is likely to benefit from the treatment

In a particular embodiment, when the percentage of myeloid cells expressing CD11b and SIRPα is over 60% in particular over 65.3% in the biological sample, the patient is likely to positively respond to a treatment of its cancer by administration of an anti-SIRPα antibody or an antigen-binding fragment thereof that inhibits the binding between SIRPα and CD47, preferably between human SIRPα and human CD47.

In a preferred embodiment, a further step of detecting the presence of Tumor Associated Macrophages, Monocytes, myeloid dendritic cells, Tumor Associates Neutrophils and/or MDSCs, in particular MDSCs, within the biological sample is performed. The presence of Tumor Associated Macrophages, Monocytes, myeloid dendritic cells, Tumor Associates Neutrophils and/or MDSCs, in particular MDSCs, is indicative that the patient is likely to positively respond to a treatment of its cancer by administration of a combination of a) an anti-SIRPα antibody or an antigen-binding fragment thereof that between SIRPα and CD47, preferably between human SIRPα and human CD47, and b) a compound that is an immune checkpoint inhibitor such as a compound that inhibits the binding between PD-1 and PD-L1, preferably between human PD-1 and human PD-L1, in particular an anti-PD-L1 antagonist antibody or an anti-PD1 antagonist antibody, more particularly an anti-PD-1 antagonist antibody or a compound that targets the lymphocyte activation gene-3 (LAG-3), in particular an antibody against lymphocyte activation gene-3. In this case, the patient is classified as likely to positively respond to a treatment of its cancer by administration of a combination of a) an anti-SIRPα antibody or an antigen-binding fragment thereof that inhibits the binding between SIRPα and CD47, preferably between human SIRPα and human CD47, and b) a compound that is an immune checkpoint inhibitor such as a compound that inhibits the binding between PD-1 and PD-L1, preferably between human PD-1 and human PD-L1, in particular an anti-PD-L1 antagonist antibody or an anti-PD1 antagonist antibody, more particularly an anti-PD-1 antagonist antibody or a compound that targets the lymphocyte activation gene-3 (LAG-3), in particular an antibody against lymphocyte activation gene-3. The biological sample provided to perform the method is preferably a biopsy issued from the microenvironment of a tumor. The markers expressed by myeloid cells can be detected by method known in the art and described here above. CD11b and SIRPα, can be detected in the biological sample by a method selected from an immunohistochemical analysis, immunoassay, a gene expression profile, a fluorescence detection, an enzymatic activity assay, a chemiluminescence detection, polymerase chain reaction, reverse-transcriptase-polymerase chain reaction, antibody binding, receptor binding arrays, target specific primers extension, ELISA, radioactive labelling.

In one aspect the invention relates to a method for treating a cancer in a patient in need thereof, comprising:
(a) determining that the patient has a cancer that exhibits a tumor whose microenvironment (TME) comprises myeloid cells that express both CD11b and SIRPα,
(b) administering an effective amount of an anti-SIRPα antibody or an antigen-binding fragment thereof that inhibits the binding between SIRPα and CD47, preferably between human SIRPα and human CD47 to said patient.

In a particular embodiment, the determination of step (a) above is a detection of myeloid cells that express both CD11b and SIRPα.

In an embodiment, the invention relates to a method for treating a cancer in a patient in need thereof, comprising:
(a) selecting a patient who has a cancer that exhibits a tumor whose microenvironment (TME) comprises myeloid cells that express both CD11b and SIRPα,
(b) administering an effective amount of an anti-SIRPα antibody or an antigen-binding fragment thereof that inhibits the binding between SIRPα and CD47, preferably between human SIRPα and human CD47 to said patient.

In an embodiment, the invention relates to a method for treating a cancer in a patient in need thereof, comprising:
(a) determining that the patient has a cancer that exhibits one or more attributes comprising:
   at least 55%, in particular at least 60%, preferably at least 65.3% of myeloid cells present within the microenvironment of a tumor, express the biological markers CD11b and SIRPα;
(b) administering an effective amount of an anti-SIRPα antibody or an antigen-binding fragment thereof that inhibits the binding between SIRPα and CD47, preferably between human SIRPα and human CD47 to said patient

In a particular embodiment, the determination of step (a) above is a detection of myeloid cells that express both CD11b and SIRPα.

In an embodiment, the invention relates to a method for treating a cancer in a patient in need thereof, comprising:
(a) selecting a patient who has a cancer that exhibits one or more attributes comprising:
   at least 55%, in particular at least 60%, preferably at least 65.3% of myeloid cells present within the microenvironment of a tumor, express the biological markers CD11b and SIRPα;
(b) administering an effective amount of an anti-SIRPα antibody or an antigen-binding fragment thereof that inhibits the binding between SIRPα and CD47, preferably between human SIRPα and human CD47 to said patient.

In the methods of the invention or in the use of the invention comprising a step of administering an effective amount of an anti-SIRPα antibody or an antigen-binding fragment thereof that inhibits the binding between SIRPα and CD47, preferably between human SIRPα and human CD47 to the treated patient, the method may comprise a final step of determining that the patient exhibits an outcome that is improved as compared to a corresponding outcome that would be observed in a reference patient that has been administered said anti-SIRPα antibody or an antigen-binding fragment thereof, wherein the reference patient has a cancer that does not exhibit said disclosed attributes.

In an embodiment, the invention relates to a method for treating cancer in a population of cancer patients in need thereof, comprising:
(a) administering an effective amount of an anti-SIRPα antibody or an antigen-binding fragment thereof that inhibits the binding between SIRPα and CD47, preferably between human SIRPα and human CD47 to patients in the population of cancer patients, which patients have a cancer that exhibits a tumor whose microenvironment (TME) comprises myeloid cells that express both CD11b and SIRPα,
(b) observing a survival probability at 15 months of at least 0.45 for patients with a high expression of SIRPα, and/or less than 0.17 for patients with a low expression of SIRPα in the population of cancer patients after administration of said anti-SIRPα antibody or an antigen-binding fragment thereof, or
   observing a survival probability at 10 months of at least 0.50 for patients with a high expression of SIRPα, and/or less than 0.40 for patients with a low expression of SIRPα in the population of cancer patients after administration of said anti-SIRPα antibody or an antigen-binding fragment thereof; or
   observing a survival probability at 20 months of at least 0.25 for patients with a high expression of SIRPα, in the population of cancer patients after administration of said anti-SIRPα antibody or an antigen-binding fragment thereof.

In a particular embodiment the invention relates to a method of treating a patient in need thereof, wherein the patient has been determined to have a cancer that exhibits one or more attributes comprising:
at least 55%, in particular at least 60% preferably at least 65.3% of myeloid cells present within the microenvironment of a tumor, express the biological markers CD11b and SIRPα;
the method comprising: administering an effective amount of an anti-SIRPα antibody or an antigen-binding fragment thereof to said patient

In another embodiment, the invention relates to a method for treating cancer in a patient in need thereof, wherein the patient has been determined to have a tumor that exhibits at least 55%, in particular at least 60% preferably at least 65.3% of myeloid cells present within the microenvironment of a tumor, express the biological markers CD11b and SIRPα;
the method comprising: administering an effective amount of an anti-SIRPα antibody or an antigen-binding fragment thereof to said patient.

In another embodiment the invention relates to a method for treating cancer in a patient in need thereof, wherein a tumor sample obtained from the patient has been determined to exhibit at least 55%, in particular at least 60%, preferably at least 65.3% of myeloid cells present within the microenvironment of a tumor, express the biological markers CD11b and SIRPα;
the method comprising: administering an effective amount of an anti-SIRPα antibody or an antigen-binding fragment thereof to said patient.

In another embodiment, the invention relates to a method for treating cancer in a patient in need thereof comprising:
(a) selecting a patient who has a tumor that exhibits at least 55%, in particular at least 60%, preferably at least 65.3% of myeloid cells present within the microenvironment of a tumor, express the biological markers CD11b and SIRPα; and
(b) administering an effective amount of an anti-SIRPα antibody or an antigen-binding fragment thereof to said patient.

In another embodiment, the invention relates to a method of treating cancer in a patient in need thereof, comprising:
(a) testing a sample from a cancer patient and determining the presence of one or more attributes comprising:
   at least 55%, in particular at least 60%, preferably at least 65.3% of myeloid cells present within the microenvironment of a tumor, express the biological markers CD11b and SIRPα; and
(b) administering an effective amount of an anti-SIRPα antibody or an antigen-binding fragment thereof to said patient.

In another embodiment, the invention relates to a method of treating a cancer patient comprising administering to a cancer patient an anti-SIRPα antibody or an antigen-binding fragment thereof, wherein the cancer patient has a tumor that has one or more attributes comprising:
at least 55%, in particular at least 60%, preferably at least 65.3% of myeloid cells present within the microenvironment of a tumor, express the biological markers CD11b and SIRPα.

In another embodiment, the invention relates to a method of categorizing a tumor of a human, comprising:
(a) testing a sample from the human to detect the presence of myeloid cells in the microenvironment of a tumor that express the biological markers CD11b and SIRPα;
(b) optionally determining the presence of at least 55%, in particular at least 60%, preferably at least 65.3% of myeloid cells present within the microenvironment of a tumor, that express the biological markers CD11b and SIRPα; and
(c) identifying the tumor as a good candidate for treatment with an anti-SIRPα antibody or an antigen-binding fragment thereof.

In any of the embodiments of methods of treatment as disclosed herein the method may further comprise the step of prescribing an anti-SIRPα antibody or an antigen-binding fragment thereof for the human.

In any of the embodiments of methods of treatment as disclosed herein the method may further comprise administering to said patient a compound that inhibits the binding between PD-1 and PD-L1, preferably between human PD-1 and human PD-L1, more preferably an anti-PD-L1 antagonist antibody or an anti-PD1 antagonist antibody, more preferably an anti-PD-1 antagonist antibody.

In an embodiment of this method the patient has been diagnosed with a SIRPα-positive cancer, a PD-1-positive cancer or a PD-L1-positive cancer, preferably a cancer with solid tumor(s) expressing or over-expressing SIRPα, PD-1 and/or PD-L1.

In any of the embodiments of methods of treatment as disclosed herein the anti-SIRPα antibody or antigen-binding fragment thereof inhibits the binding between SIRPα and CD47, preferably between human SIRPα and human CD47.

In any of the embodiments of methods of treatment as disclosed herein the anti-SIRPα antibody or antigen-binding fragment thereof may comprise:
i) A heavy chain variable domain comprising the amino acid sequence set forth in SEQ ID NO: 3, or in SEQ ID NO: 4, or in SEQ ID NO: 5; or in SEQ ID NO: 6; or in SEQ ID NO: 7; or in SEQ ID NO: 8; and
ii) A light chain variable domain comprising the amino acid sequence set forth in SEQ ID NO: 9 or in SEQ ID NO: 10, or
i′) a heavy chain variable domain comprising:
   a heavy chain CDR1 (HCDR1) domain comprising or consisting of the amino acid sequence set forth in SEQ ID No. 15; and
   a heavy chain CDR2 (HCDR2) domain comprising or consisting of the amino acid sequence set forth in SEQ ID No. 16 or SEQ ID No. 17; and
   a heavy chain CDR3 (HCDR3) domain comprising or consisting of the amino acid sequence set forth in SEQ ID No. 18 or SEQ ID No. 19 or SEQ ID No. 20 or SEQ ID No. 21; and
ii′) a light chain variable domain comprising:
   a light chain CDR1 (LCDR1) domain comprising or consisting of the amino acid sequence set forth in SEQ ID No. 22; and
   a light chain CDR2 (LCDR2) domain comprising or consisting of the amino acid sequence set forth in SEQ ID No. 23; and
   a light chain CDR3 (LCDR3) domain comprising or consisting of the amino acid sequence set forth in SEQ ID No. 24.
wherein preferably the anti-SIRPα antibody or antigen-binding fragment thereof comprises the heavy chain variable domain comprising the amino acid sequence set forth in SEQ ID NO: 8 and the light chain variable domain comprising the amino acid sequence set forth in SEQ ID NO: 10.

In a particular embodiment of any of these methods of treatment, the anti-SIRPα antibody or antigen-binding fragment thereof comprises:
i′) a heavy chain comprising the amino acid sequence set forth in SEQ ID NO: 11, and a light chain comprising of the amino acid sequence set forth in SEQ ID NO: 12; or
ii′) a heavy chain comprising the amino acid sequence set forth in SEQ ID NO: 13, and a light chain comprising of the amino acid sequence set forth in SEQ ID NO: 12.
iii′) a heavy chain variable domain comprising:
   a heavy chain CDR1 (HCDR1) domain comprising or consisting of the amino acid sequence set forth in SEQ ID No. 15; and
   a heavy chain CDR2 (HCDR2) domain comprising or consisting of the amino acid sequence set forth in SEQ ID No. 17; and
   a heavy chain CDR3 (HCDR3) domain comprising or consisting of the amino acid sequence set forth in SEQ ID No. 21; and
a light chain variable domain comprising:
   a light chain CDR1 (LCDR1) domain comprising or consisting of the amino acid sequence set forth in SEQ ID No. 22; and
   a light chain CDR2 (LCDR2) domain comprising or consisting of the amino acid sequence set forth in SEQ ID No. 23; and
   a light chain CDR3 (LCDR3) domain comprising or consisting of the amino acid sequence set forth in SEQ ID No. 24.

In another embodiment of the methods of treatment as disclosed herein, the anti-SIRPα antibody or antigen-binding fragment thereof comprises an anti-SIRPα antibody as defined herein.

In a particular embodiment of the herein disclosed methods of treating a patient or a cancer, said myeloid cells comprise Tumor Associated Macrophages, Monocytes, MDSCs, myeloid dendritic cells, and/or Tumor Associates Neutrophils.

In an embodiment of the invention, it is provided a method of treating a patient having a cancer that is likely to benefit from a treatment with an anti-SIRPα antibody or an antigen-binding fragment thereof that inhibits the binding between SIRPα and CD47, preferably between human SIRPα and human CD47, wherein the patient is in need of treatment of a cancer with a solid tumor, the method comprising the steps of:
administering an effective amount of an anti-SIRPα antibody or an antigen-binding fragment thereof that inhibits the binding between SIRPα and CD47, preferably between human SIRPα and human CD47, to the patient in need thereof;
wherein the patient has been determined to have myeloid cells expressing CD11b and SIRPα biomarkers in the microenvironment of the patient's tumor (TME).

The anti-SIRPα antibody or an antigen-binding fragment can correspond to any anti-SIRPα antibody or an antigen-binding fragment disclosed or reference herein, in particular:
comprises the heavy chain variable domain comprising the amino acid sequence set forth in SEQ ID NO: 8 and the light chain variable domain comprising the amino acid sequence set forth in SEQ ID NO: 10; or
comprises: a heavy chain variable domain comprising:
   a heavy chain CDR1 (HCDR1) domain comprising or consisting of the amino acid sequence set forth in SEQ ID No. 15; and
   a heavy chain CDR2 (HCDR2) domain comprising or consisting of the amino acid sequence set forth in SEQ ID No. 17; and
   a heavy chain CDR3 (HCDR3) domain comprising or consisting of the amino acid sequence set forth in SEQ ID No. 21; and
a light chain variable domain comprising:
   a light chain CDR1 (LCDR1) domain comprising or consisting of the amino acid sequence set forth in SEQ ID No. 22; and
   a light chain CDR2 (LCDR2) domain comprising or consisting of the amino acid sequence set forth in SEQ ID No. 23; and
   a light chain CDR3 (LCDR3) domain comprising or consisting of the amino acid sequence set forth in SEQ ID No. 24, or
comprises a heavy chain comprising the amino acid sequence set forth in SEQ ID NO: 11, and a light chain comprising of the amino acid sequence set forth in SEQ ID NO: 12; or a heavy chain comprising the amino acid sequence set forth in SEQ ID NO: 13, and a light chain comprising of the amino acid sequence set forth in SEQ ID NO: 12.

The anti-SIRPα antibody or an antigen-binding fragment can correspond to the anti-SIRPα antibody or an antigen-binding fragment disclosed in WO 2020/099653, WO 2019/023347, WO 2022/254379, WO 2020/068752, WO 2021/226576, WO2021174127, WO 2019/226973, WO 2018/190719, US20210347908, S20210347908, US20210347908, WO0066159, WO0140307, WO200140307, WO2009131453, WO2013056352, WO2014149477, WO2014186761, WO2015138600, WO2016063233, WO2016205042, WO2017178653, WO2018008470, WO2018026600, WO2018057669, WO2018107058, WO2018141964, WO2018160739, WO2018190719, WO2018210793, WO2019023347, WO2019183266, WO2019200462, WO2019226973, WO2020006374, WO2020013170, WO2020033646, WO2020068752, WO2020102422, WO2020099653, WO2020180811, WO2020247820, WO2021022044, WO2021032078, WO2021076908, WO2021129697, WO2021174127, WO2021185273, CN111635458, WO2021222746, WO2021226576, WO2021226591, CN113735973, CN111995682, CN112010979, or CN112574310, in particular the anti-SIRPα antibody or an antigen-binding fragment disclosed in any one of these publications and referenced herein.

In a particular embodiment of the herein disclosed methods of treating a patient or a tumor, the patient has at least 55%, in particular at least 60%, preferably at least 65.3% of its myeloid cells present within the microenvironment of a tumor, preferably Tumor Associated Macrophages, Monocytes, MDSCs, myeloid dendritic cells, and/or Tumor Associates Neutrophils, that express the biological markers CD11b and SIRPα.

In a particular embodiment of the herein disclosed methods of treating a patient or a tumor, the patient has Tumor Associated Macrophages, Monocytes, myeloid dendritic cells, Tumor Associates Neutrophils and/or MDSCs, in particular MDSCs, within the microenvironment of the tumor.

In a particular embodiment of the herein disclosed methods of treating a patient or a tumor, the patient has liquid or solid cancer, preferably a cancer with advanced solid tumor(s), more particularly an adrenal cancer, a biliary tree cancer, a breast cancer, a cervical cancer, a colorectal cancer, an endometrial cancer, a gastrointestinal cancer, a head and neck cancer, a kidney cancer, a liver cancer, a lung cancer, a melanoma, a non-small cells lung cancer, an ovarian cancer, a hepatocellular carcinoma, a pancreas cancer, a parotid cancer, a prostate cancer or an uterine cancer, more particularly an ovarian cancer, a breast cancer in particular a Triple Negative Breast cancer, lung cancer more particularly Non-Small Cells Lung Cancer (NSCLC), cervical cancer, and colorectal cancer, most particularly Non-Small Cell Lung Cancer (NSCLC), cervical cancer, or colorectal cancer.

In another embodiment, the invention concerns a method for treating cancer in a patient, wherein the patient has been diagnosed with a cancer, the method comprising the following steps:
Providing a biological sample, in particular a blood sample, a plasma sample, a serum sample, a biopsy and/or a tumor sample, previously obtained from the patient, more particularly a biological sample previously obtained from the patient wherein the sample is from the microenvironment of the patient's tumor (TME) and comprises myeloid cells, in particular the sample is a biopsy of the microenvironment of the patient's tumor
Measuring the presence of myeloid cells, in particular Tumor Associated Macrophages, Monocytes, myeloid dendritic cells, MDSCs, and/or Tumor Associates Neutrophils, expressing CD11b and SIRPα in the biological sample,
When the percentage of myeloid cells expressing CD11b and SIRPα is over 60% in particular over 65.3% in the biological sample, administering to the patient a therapeutically effective amount of an anti-SIRPα antibody or an antigen-binding fragment thereof that inhibits the binding between SIRPα and CD47, preferably between human SIRPα and human CD47.

In a particular embodiment, the invention concerns a method for treating cancer in a patient, wherein the patient has been diagnosed with a cancer, the method comprising the following steps:
Providing a biological sample, in particular a blood sample, a plasma sample, a serum sample, a biopsy and/or a tumor sample, previously obtained from the patient,
Measuring the presence of myeloid cells, in particular Tumor Associated Macrophages, Monocytes, myeloid dendritic cells, MDSCs, and/or Tumor Associates Neutrophils, expressing CD11b and SIRPα in the biological sample,
optionally measuring the presence of Tumor Associated Macrophages, Monocytes, myeloid dendritic cells, MDSCs, and/or Tumor Associates Neutrophils, in particular Myeloid-Derived Suppressor Cells (MDSCs), within the biological sample,
When the percentage of myeloid cells expressing CD11b and SIRPα is over 65.3% in the biological sample, and when Tumor Associated Macrophages, Monocytes, myeloid dendritic cells, MDSCs, and/or Tumor Associates Neutrophils, in particular Myeloid-Derived Suppressor Cells (MDSCs), are detected within the sample, administrate to the patient a combination therapy comprising a) an anti-SIRPα antibody or an antigen-binding fragment thereof that inhibits the binding between SIRPα and CD47, preferably between human SIRPα and human CD47 and b) with a compound that inhibits the binding between PD-1 and PD-L1, preferably between human PD-1 and human PD-L1, in particular an anti-PD-L1 antagonist antibody or an anti-PD1 antagonist antibody, more particularly an anti-PD-1 antagonist antibody.

In a particular embodiment, the patient has a cancer with solid tumors, in particular advanced solid tumors. In another embodiment the invention concerns a method for treating cancer in a patient, wherein the patient is treated for ovarian cancer, pancreas cancer, Vater's ampulla cancer, Micro Satellite Stable (MSS) cancer, Micro Satellite Instable (MSI) cancer, colorectal cancer, in particular MSI and MSS colorectal cancers, fibrolamellar cancer, breast cancer, endocrinal cancer, hepatocellular carcinoma, melanoma, kidney cancer, lung cancer, in particular non-small cell lung cancer (NSCLC), head and neck cancer, in particular head and neck squamous cell carcinoma (HNSCC), gastric cancer and hepatocellular carcinoma cancer.

In another embodiment, the invention concerns a method for treating cancer in a patient, wherein the patient has been diagnosed with a SIRPα-positive cancer, a PD-1-positive cancer or a PD-L1-positive cancer, in particular a cancer with solid tumors expressing or over-expressing SIRPα, PD-1 and/or PD-L1.

In another embodiment the invention concerns a method for treating cancer in a patient, who was not previously treated with an anti-PD-L1 antibody or an anti-PD-1 antibody, in particular an anti-PD-1 antagonist antibody or an anti-PD-L1 antagonist antibody.

In another embodiment the invention concerns a method for treating cancer in a patient, wherein the patient has, prior to the use, not been treated with an anti-SIRPα antibody.

In another embodiment the invention concerns a method for treating cancer in a patient who exhibited disease progression in response to a previous treatment with an anti-PD-L1 antibody or an anti-PD-1 antibody, in particular an anti-PD-1 antagonist antibody or an anti-PD-L1 antagonist antibody.

In another embodiment the invention concerns a method for sensitizing a patient with a cancer to a treatment with an anti-PD-1 antagonist antibody or with an anti-PD-L1 antibody, the anti-SIRPα antibody or antigen-binding fragment thereof being administered during a first period, and the anti-PD-1 antagonist antibody or the anti-PD-L1 antibody being administered during a second, subsequent, period of time, wherein the anti-PD-1 antagonist antibody or the anti-PD-L1 antibody is not administered during the first period of time.

In another embodiment, the invention concerns a method for treating cancer in a patient, wherein the anti-SIRPα antibody or antigen-binding fragment thereof comprises:
i) A heavy chain variable domain comprising the amino acid sequence set forth in SEQ ID No: 3, or in SEQ ID No: 4, or in SEQ ID No: 5; or in SEQ ID No: 6; or in SEQ ID No: 7; or in SEQ ID No: 8; and
   A light chain variable domain comprising the amino acid sequence set forth in SEQ ID No: 9 or in SEQ ID No: 10.
ii) a heavy chain variable domain comprising:
   a. a heavy chain CDR1 (HCDR1) domain comprising or consisting of the amino acid sequence set forth in SEQ ID No. 15; and
   b. a heavy chain CDR2 (HCDR2) domain comprising or consisting of the amino acid sequence set forth in SEQ ID No. 16 or SEQ ID No. 17; and
   c. a heavy chain CDR3 (HCDR3) domain comprising or consisting of the amino acid sequence set forth in SEQ ID No. 18 or SEQ ID No. 19 or SEQ ID No. 20 or SEQ ID No. 21; and
a light chain variable domain comprising:
   d. a light chain CDR1 (LCDR1) domain comprising or consisting of the amino acid sequence set forth in SEQ ID No. 22; and
   e. a light chain CDR2 (LCDR2) domain comprising or consisting of the amino acid sequence set forth in SEQ ID No. 23; and
   f. a light chain CDR3 (LCDR3) domain comprising or consisting of the amino acid sequence set forth in SEQ ID No. 24.
      in particular comprises the heavy chain variable domain comprising the amino acid sequence set forth in SEQ ID No: 8 and the light chain variable domain comprising the amino acid sequence set forth in SEQ ID No: 10.

In another embodiment, the invention concerns a method for treating cancer in a patient by administering an anti-SIRPα antibody or antigen-binding fragment thereof that comprises the heavy chain variable domain comprising the amino acid sequence set forth in SEQ ID No: 8 and the light chain variable domain comprising the amino acid sequence set forth in SEQ ID No: 10.

In another embodiment, the invention concerns a method for treating cancer in a patient by administering an anti-SIRPα antibody that comprises the heavy chain variable domain comprising the amino acid sequence set forth in SEQ ID No: 8 and the light chain variable domain comprising the amino acid sequence set forth in SEQ ID No: 10.

In another embodiment, the invention concerns a method for treating cancer in a patient by administering an anti-SIRPα antibody or antigen-binding fragment thereof that comprises the heavy chain comprising the amino acid sequence set forth in SEQ ID No: 11 and the light chain comprising the amino acid sequence set forth in SEQ ID No: 12.

In another embodiment, the invention concerns a method for treating cancer in a patient by administering an anti-SIRPα antibody that comprises the heavy chain comprising the amino acid sequence set forth in SEQ ID No: 13 and the light chain comprising the amino acid sequence set forth in SEQ ID No: 12.

### DESCRIPTION OF THE FIGURES

FIG. 1 illustrates the absence of correlation between the patient overall survival and CD47 expression in tumor cells. Patients with a low expression of CD47 (as compared to the median expression of CD47 in pools of patients) is illustrated by the black dotted line. Patients with a high expression of CD47 (as compared to the median expression of CD47 in pools of patients) is illustrated by the light grey line. Continuous lines correspond to the mean of either all high-expressing CD47 patients or all low-expressing CD47 patients. Time is in months. Survival probability is assessed by comparing over time the number of patients alive to the overall number of patients at the beginning of the study. A) mean of all treated patients (either by monotherapy of anti-SIRPα antibody or by a combination therapy of an anti-SIRPα antibody (comprising the heavy chain variable domain of SEQ ID No. 8 and the light chain variable domain of SE ID No. 10; this anti-SIRPα antibody has been used in all experiments disclosed herein) and a PD1 inhibitor (ezabenlimab, an anti-PD1 antagonist antibody). B) mean of patients treated by a monotherapy of an anti-SIRPα antibody.

FIG. 2 illustrates the absence of correlation between the patient overall survival and SIRPα expression in tumor cells. Patients with a low expression of SIRPα (as compared to the median expression of SIRPα in pools of patients) is illustrated by the black dotted line. Patients with a high expression of SIRPα (as compared to the median expression of SIRPα in pools of patients) is illustrated by the light grey line. Continuous lines correspond to the mean of either all high-expressing SIRPα patients or all low-expressing SIRPα patients. Time is in months. Survival probability is assessed by comparing over time the number of patients alive to the overall number of patients at the beginning of the study. A) mean of all treated patients (either by monotherapy with an anti-SIRPα antibody or by a combination therapy with an anti-SIRPα antibody and a PD1 inhibitor, as defined in the legend of FIG. 1. B) mean of patients treated by a monotherapy with an anti-SIRPα antibody as defined in the legend of FIG. 1.

FIG. 3 illustrates the correlation between the patient overall survival and SIRPα expression in CD11b-positive myeloid cells issued from the microenvironment of a tumor. A and B. Patients with a low expression of SIRPα (lower than 65.3% of CD11b+ SIRPa+ myeloid cells) are represented by the black dotted line. Patients with a high expression of SIRPα (at least 65.3% of CD11b+ SIRPa+ myeloid cells) are represented by the light grey line. A) mean of all treated patients (either by monotherapy with an anti-SIRPα antibody or by a combination therapy with an anti-SIRPα antibody and a PD1 inhibitor, as defined in the legend of FIG. 1. B) mean of patients treated by a monotherapy with an anti-SIRP antibody as defined in the legend of FIG. 1. C and D. Patients with a low expression of SIRPα (lower than 60% of CD11b+ SIRPa+ myeloid cells) are represented by the black dotter line. Patients with a high expression of SIRPα (at least 60% of CD11b+ SIRPa+ myeloid cells) are represented by the light grey line. C) mean of all treated patients (either by monotherapy with an anti-SIRPα antibody or by a combination therapy with an anti-SIRPα antibody and a PD1 inhibitor, as defined in the legend of FIG. 1. D) mean of patients treated by a monotherapy with an anti-SIRPα antibody as defined in the legend of FIG. 1. E and F. Patients with a low expression of SIRPα (lower than 55% of CD11b+ SIRPα+ myeloid cells) are represented by the black dotted line. Patients with a high expression of SIRPα (at least 55% of CD11b+ SIRPa+ myeloid cells) are represented by the light grey line. Continuous lines correspond to the mean of either all high-expressing SIRPα patients or all low-expressing SIRPα patients. Dotted lines correspond to the standard deviation for each group of patients. Survival probability is assessed by comparing over time the number of patients alive to the overall number of patients at the beginning of the study. E) mean of all treated patients (either by monotherapy with an anti-SIRPα antibody or by a combination therapy with an anti-SIRPα antibody and a PD1 inhibitor as defined in the legend of FIG. 1. F) mean of patients treated by a monotherapy with an anti-SIRPα antibody as defined in the legend of FIG. 1.

FIG. 4 illustrates the correlation between the gene expression profile in patient positively responding to a combination therapy (abscissa and left part) with an anti-SIRPα antibody and a PD1 inhibitor, and the known gene expression profile in MDSCs (ordinate and right part).

### EXAMPLES

### Materials and Methods

Treatment outcomes, as Overall Survival (OS) and Progression Free Survival (PFS) are collected from the iCRF (case report form) documents.

Features of interest as CD47 expression in tumor cells at baseline, SIRPα expression in CD11b+ myeloid cells are evaluated by Immunohistochemistry (IHC) using the Veracyte BrightPlex® technology and a Leica Bond RX for successive staining. Each tissue slide is scanned with the Nanozoomer XR/x20. Samples before treatment are split in High or Low expressors based on the level expression of these features compared to the median of expression of the study population.

The Kaplan Meier Curve is used to estimate the survival and is calculated with the Rpackage survival. The statistical test used is the log-rank test to compare the survival of High and Low expressor groups. The log-rank test addresses the hypothesis that there are no differences between the populations being studied in the probability of an event at any time point and the differences are considered significant when pvalue <0.05.

### Example 1—Expression of CD47, SIRPα and CD11b in Tumor Cells and Immune Cells Issued from the TME of Patients Having a Cancer

Patient's status has been assessed at baseline for a biomarker of response to the anti-SIRPα antibody or an antigen-binding fragment thereof

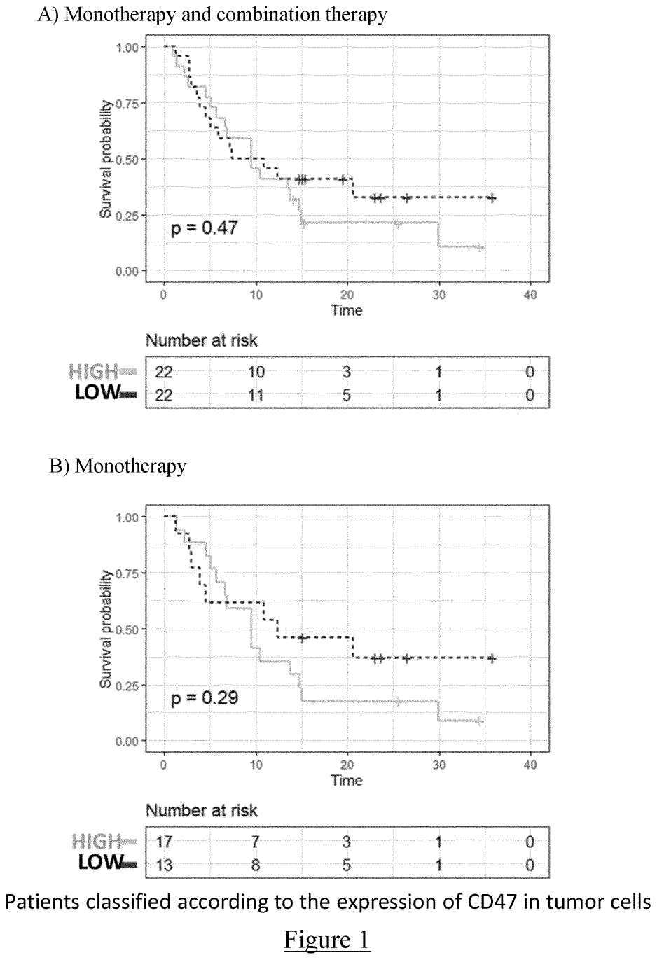

FIG. 1 illustrates that classifying the patients by the overall expression of CD47 in tumor cells does not allow to discriminate the patients who are benefiting from the treatment with an anti-SIRPα antibody in regard to Overall Survival from those patients that are not benefiting from the treatment despite being treated with the same antibody. Whether the patients are treated by a monotherapy of an anti-SIRPαantibody or a combination therapy of an anti-SIRPα antibody and a PD1 inhibitor, assessing the expression of CD47 in tumor cells is not suitable to assess if a patient is likely to benefit from a treatment with an anti-SIRPα antibody, either alone or in combination, in particular in combination with a PD1 inhibitor.

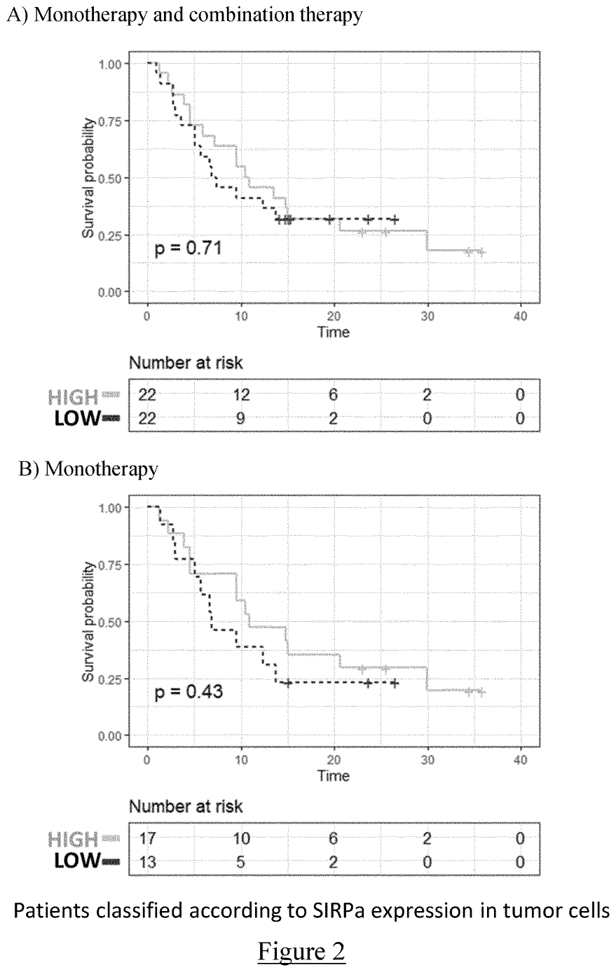

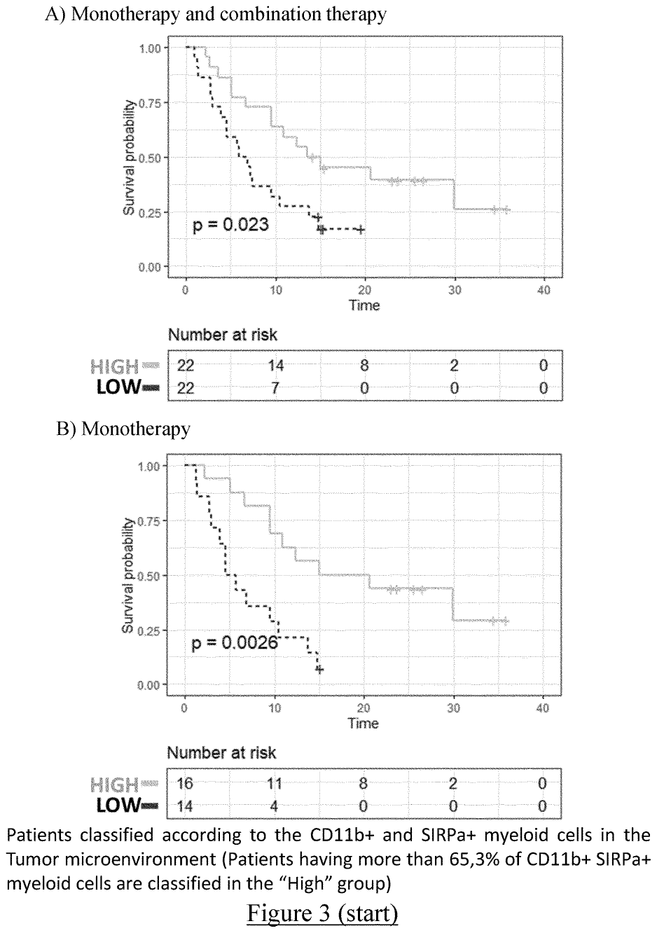

FIG. 2 illustrates that classifying the patients by the overall expression of SIRPα in tumor cells does not allow to discriminate the patients who are benefiting from the treatment with an anti-SIRPα antibody in regard to Overall Survival from the patients that are not benefiting from the treatment despite being treated with the same antibody. Whether the patients are treated by a monotherapy of an anti-SIRPα antibody or a combination therapy of an anti-SIRPα antibody and a PD1 inhibitor, assessing the expression of CD47 in tumor cells is not suitable to assess if a patient is likely to benefit from a treatment with an anti-SIRPα antibody, either alone or in combination, in particular in combination with a PD1 inhibitor.

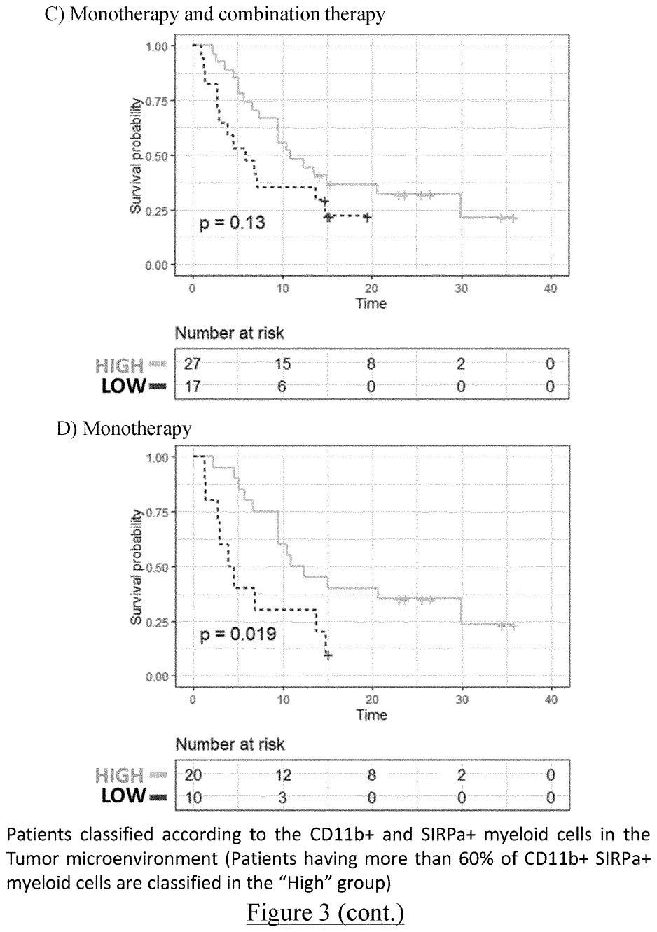

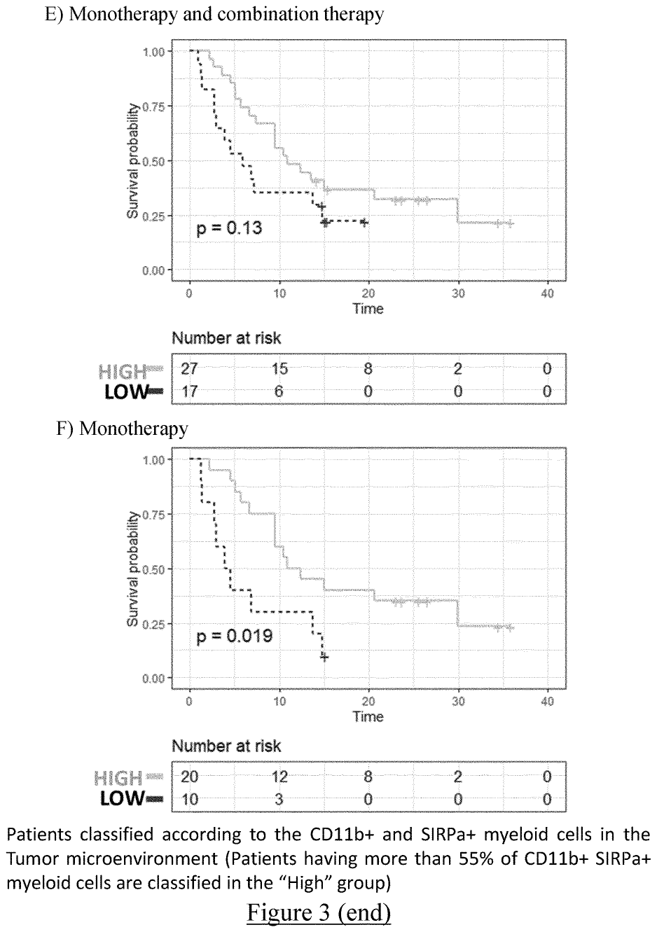

FIG. 3 illustrates that classifying the patients according to the presence of CD11b+ SIRPα+ myeloid cells in the tumor microenvironment allow to discriminate the patients who are benefiting from the treatment with an anti-SIRPα antibody (i.e., whose disease regresses or does not progress or progress more slowly) from those patients that are not benefiting from the treatment despite being treated with the same antibody. As illustrated in FIGS. 3A and 3B, the patients who have at least 65.3% CD11b+ SIRPa+ myeloid cells survive longer than patients who have less than 65.3% of CD11b+ SIRPα+ myeloid cells: after 30 months of treatment, 25% of the patients who have at least 65.3% CD11b+ SIRPa+ myeloid cells are still alive. This classification of the patients is valid when patients are treated by monotherapy with an anti-SIRPα antibody and when patients are treated by a combination therapy with an anti-SIRPα antibody, in particular in combination with a PD1 inhibitor.

FIGS. 3C, 3D, 3E and 3E illustrate that the patients who have at least 60% (FIGS. 3C and 3D) or 55% (FIGS. 3E and 3F) CD11b+ SIRPa+ myeloid cells survive longer than patients who have less than 60% or 55% of CD11b+ SIRPα+ myeloid cells: after 20 months of treatment, 25% of the patients who have at least 60% or 55% CD11b+ SIRPa+ myeloid cells are still alive. This classification of the patients is valid when patients are treated by monotherapy with an anti-SIRPα antibody and when patients are treated by a combination therapy with an anti-SIRPα antibody, in particular in combination with a PD1 inhibitor. The overall survival curve of patients who have more than 65.3% of CD11b+ SIRPα+ myeloid cells decreases more slowly than the overall survival curve of patients who have more than 60% or 55% of CD11b+ SIRPα+ myeloid cells, in patients treated by a monotherapy or a combination therapy with an anti-SIRPα antibody. Overall survival of the patients who have more than 65.3% of CD11b+ SIRPα+ myeloid cells is above the overall survival of patients having a lower percentage of CD11b+ α+ myeloid cells after 20 months of treatment.

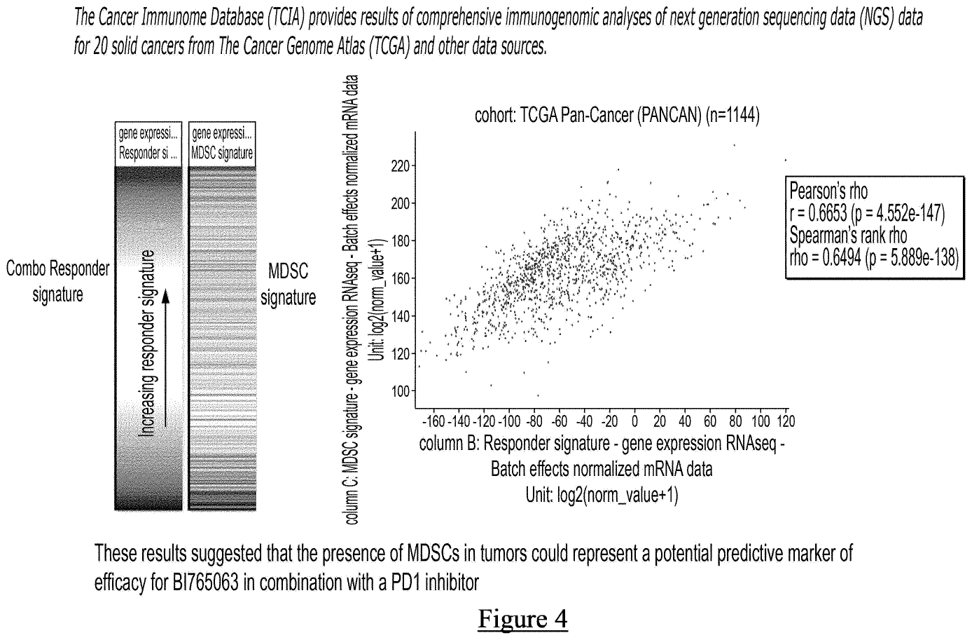

Further, the overall survival curve of patients who have more than 65.3% of CD11b+ SIRPα+ myeloid cells decreases more slowly than the overall survival curve of patients who have less than 60% of CD11b+ SIRPα+ myeloid cells. FIG. 4 illustrates that the gene expression profile in immune cells present within the microenvironment of the tumor of a patient treated with a combination of an anti-SIRPα antibody and a PD1 inhibitor and who positively respond to this treatment is similar to the gene expression profile of MDSCs. Thus, these results show that the presence of MDSCs within the microenvironment of the tumor is a potential predictive marker of efficacy of the combination therapy with an anti-SIRPα antibody and a PD1 inhibitor.

The result of predictivity is particularly advantageous for a combination therapy comprising the administration of both an anti-SIRPα antibody and an anti PD1.

1. A composition comprising an anti-SIRPα compound, in particular an anti-SIRPα antibody or an antigen-binding fragment thereof, that inhibits the binding between SIRPα and CD47, preferably between human SIRPα and human CD47, for use in the treatment of cancer in a patient, wherein the cancer is characterized by a tumor whose microenvironment (TME) comprises myeloid cells that express both CD11b and SIRPα, and wherein the patient has, before the treatment and/or during the treatment, at least 55% of its myeloid cells present within the TME that express CD11b and SIRPα, in particular at least 60%, more particularly at least 65.3%.

2. The composition for use according to claim 1 wherein the myeloid cells from the TME are issued from a biological sample previously obtained from the TME of the patient.

3. The composition for use according to claim 1, wherein the microenvironment of the tumor of the patient comprises Tumor Associated Macrophages, Monocytes, myeloid dendritic cells, MDSCs, and/or Tumor Associates Neutrophils, in particular, MDSCs that express the biological markers CD11b and SIRPα.

4. The composition for use according to claim 1, wherein the patient has myeloid cells within the microenvironment of the tumor.

5. The composition for use according to claim 1, for treatment of a patient who has a liquid cancer or a solid cancer, in particular a cancer with advanced solid tumor(s), more particularly an adrenal cancer, a biliary tree cancer, a breast cancer, a cervical cancer, a colorectal cancer, an endometrial cancer, a gastrointestinal cancer, a head and neck cancer, a kidney cancer, a liver cancer, a lung cancer, a melanoma, a non-small cells lung cancer, an ovarian cancer, a hepatocellular carcinoma, a pancreas cancer, a parotid cancer, a prostate cancer or an uterine cancer, more particularly an ovarian cancer, a breast cancer in particular a Triple Negative Breast cancer, lung cancer more particularly Non-Small Cells Lung Cancer (NSCLC), cervical cancer, and colorectal cancer, most particularly Non-Small Cell Lung Cancer (NSCLC), cervical cancer, or colorectal cancer.

6. The composition for use according to claim 1, wherein the composition is administered in a combination treatment with an immune checkpoint inhibitor of the interaction between tumor cell and myeloid cells, in particular with a compound selected from the groups consisting of an anti-PD-L1, anti-PD-1, anti_LAG-3, anti-CTLA4, anti-CD137, anti-CD2, anti-CD28, anti-CD40, anti-HVEM, anti-BTLA, anti-CD160, anti-TIGIT, anti-TIM-1/3, anti-LAG-3, anti-2B4, anti-VISTA, anti-OX40, anti-CD40 agonist, CD40-L, TLR agonists, anti-ICOS, ICOS-L, STING agonist, IDO inhibitor, oncolytic virus agonists, and B-cell receptor agonists, more particularly a compound that inhibits the binding between PD-1 and PD-L1, preferably between human PD-1 and human PD-L1, in particular an anti-PD-L1 antagonist antibody or an anti-PD1 antagonist antibody, more preferably an anti-PD-1 antagonist antibody or with a compound that targets the lymphocyte activation gene-3 (LAG-3), in particular an antibody against lymphocyte activation gene-3.

7. The composition for use according to claim 5, wherein the patient has been diagnosed with a SIRPα-positive cancer, a PD-1-positive cancer or a PD-L1-positive cancer, preferably a cancer with solid tumor(s) expressing or over-expressing SIRPα, PD-1 and/or PD-L1.

8. The composition for use according to claim 1, wherein CD11b and SIRPα are measured by ImmunoHistoChemistry (IHC).

9. The composition for use according to claim 1, wherein the anti-SIRPα antibody or antigen-binding fragment thereof comprises:
a. A heavy chain variable domain comprising the amino acid sequence set forth in SEQ ID No: 3, or in SEQ ID No: 4, or in SEQ ID No: 5; or in SEQ ID No: 6; or in SEQ ID No: 7; or in SEQ ID No: 8; and
b. A light chain variable domain comprising the amino acid sequence set forth in SEQ ID No: 9 or in SEQ ID No: 10,
wherein preferably the anti-SIRPα antibody or antigen-binding fragment thereof comprises the heavy chain variable domain comprising the amino acid sequence set forth in SEQ ID No: 8 and the light chain variable domain comprising the amino acid sequence set forth in SEQ ID No: 10.

10. The composition for use according to claim 1, which comprises an anti-SIRPα antibody that comprises:
i′) a heavy chain comprising the amino acid sequence set forth in SEQ ID No: 11, and a light chain comprising of the amino acid sequence set forth in SEQ ID No. 12; or
ii′) a heavy chain comprising the amino acid sequence set forth in SEQ ID No: 13, and a light chain comprising of the amino acid sequence set forth in SEQ ID No. 12.

11. A method of assessing a patient's status for biomarkers of response to a treatment with an anti-SIRPα antibody or an antigen-binding fragment thereof that inhibits the binding between SIRPα and CD47, preferably between human SIRPα and human CD47, wherein the patient is in need of treatment of a cancer with a liquid tumor or solid tumor, the method comprising:
providing a biological sample previously obtained from the patient wherein the sample is from the microenvironment of the patient's tumor (TME) and comprises myeloid cells, in particular the sample is a biopsy of the microenvironment of the patient's tumor,
determining the presence in the biological sample of myeloid cells expressing CD11b and SIRPα biomarkers.

12. A method for determining if a patient having a cancer is likely to benefit from a treatment with an anti-SIRPα antibody or an antigen-binding fragment thereof that inhibits the binding between SIRPα and CD47, preferably between human SIRPα and human CD47, wherein the patient is in need of treatment of a cancer with a solid tumor, the method comprising the steps of:
providing a biological sample previously obtained from the patient wherein the sample is from the microenvironment of the patient's tumor (TME) and comprises myeloid cells, in particular the sample is a biopsy of the microenvironment of the patient's tumor,
determining the presence in the biological sample of myeloid cells expressing CD11b and SIRPα biomarkers,
if myeloid cells expressing CD11b and SIRPα biomarkers are present in the biological sample, the patient is likely to benefit from the treatment.

13. The method according to claim 11, wherein when the percentage of myeloid cells expressing CD11b and SIRPα is at least 55%, in particular at least 60%, in the biological sample, the patient is as likely to positively respond to a treatment of its cancer by administration of an anti-SIRPα antibody or an antigen-binding fragment thereof that inhibits the binding between SIRPα and CD47, preferably between human SIRPα and human CD47.

14. The method according to claim 11, wherein the patient has a liquid cancer or a solid cancer, in particular a cancer with advanced solid tumor(s), more particularly an adrenal cancer, a biliary tree cancer, a breast cancer, a cervical cancer, a colorectal cancer, an endometrial cancer, a gastrointestinal cancer, a head and neck cancer, a kidney cancer, a liver cancer, a lung cancer, a melanoma, a non-small cells lung cancer, an ovarian cancer, a hepatocellular carcinoma, a pancreas cancer, a parotid cancer, a prostate cancer or an uterine cancer, more particularly an ovarian cancer, a breast cancer in particular a Triple Negative Breast cancer, lung cancer more particularly Non-Small Cells Lung Cancer (NSCLC), cervical cancer, and colorectal cancer, most particularly Non-Small Cell Lung Cancer (NSCLC), cervical cancer, or colorectal cancer.

15. The method according to claim 11, further comprising a step of detecting the presence of Tumor Associated Macrophages, Monocytes, myeloid dendritic cells, MDSCs, and/or Tumor Associates Neutrophils, in particular MDSCs, within the biological sample, the presence of Tumor Associated Macrophages, Monocytes, myeloid dendritic cells, MDSCs, and/or Tumor Associates Neutrophils, in particular MDSCs, being indicative that the patient is likely to positively respond to a treatment of its cancer by administration of a combination of a) an anti-SIRPα antibody or an antigen-binding fragment thereof that inhibits the binding between SIRPα and CD47, preferably between human SIRPα and human CD47, and b) a compound that is an immune checkpoint inhibitor, in particular a compound that inhibits the binding between PD-1 and PD-L1, preferably between human PD-1 and human PD-L1, in particular an anti-PD-L1 antagonist antibody or an anti-PD1 antagonist antibody, more particularly an anti-PD-1 antagonist antibody or a compound that targets the lymphocyte activation gene-3 (LAG-3), in particular an antibody against lymphocyte activation gene-3.

16. The method according to claim 11, wherein the patient is likely to positively respond to a treatment of its cancer by administration of a combination of a) an anti-SIRPα antibody or an antigen-binding fragment thereof that inhibits the binding between SIRPα and CD47, in particular between human SIRPα and human CD47, and b) a compound that inhibits the binding between PD-1 and PDL-1, preferably between human PD-1 and human PD-L1, in particular an anti-PD-L1 antagonist antibody or an anti-PD1 antagonist antibody, more particularly an anti-PD-1 antagonist antibody or a compound that targets the lymphocyte activation gene-3 (LAG-3), in particular an antibody against lymphocyte activation gene-3.

17. The method according to claim 11, wherein CD11b and SIRPα are detected in the biological sample by a method selected from an immunoassay, in particular ImmunoHistoChemistry, a gene expression profile, a fluorescence detection, an enzymatic activity assay, a chemiluminescence detection, polymerase chain reaction, reverse-transcriptase-polymerase chain reaction, antibody binding, receptor binding arrays, target specific primers extension, ELISA, radioactive labelling.

18. The method according to claim 12, wherein when the percentage of myeloid cells expressing CD11b and SIRPα is at least 55%, in particular at least 60%, in the biological sample, the patient is as likely to positively respond to a treatment of its cancer by administration of an anti-SIRPα antibody or an antigen-binding fragment thereof that inhibits the binding between SIRPα and CD47, preferably between human SIRPα and human CD47.

19. The method according to claim 12, wherein the patient has a liquid cancer or a solid cancer, in particular a cancer with advanced solid tumor(s), more particularly an adrenal cancer, a biliary tree cancer, a breast cancer, a cervical cancer, a colorectal cancer, an endometrial cancer, a gastrointestinal cancer, a head and neck cancer, a kidney cancer, a liver cancer, a lung cancer, a melanoma, a non-small cells lung cancer, an ovarian cancer, a hepatocellular carcinoma, a pancreas cancer, a parotid cancer, a prostate cancer or an uterine cancer, more particularly an ovarian cancer, a breast cancer in particular a Triple Negative Breast cancer, lung cancer more particularly Non-Small Cells Lung Cancer (NSCLC), cervical cancer, and colorectal cancer, most particularly Non-Small Cell Lung Cancer (NSCLC), cervical cancer, or colorectal cancer.

20. The method according to claim 12, further comprising a step of detecting the presence of Tumor Associated Macrophages, Monocytes, myeloid dendritic cells, MDSCs, and/or Tumor Associates Neutrophils, in particular MDSCs, within the biological sample, the presence of Tumor Associated Macrophages, Monocytes, myeloid dendritic cells, MDSCs, and/or Tumor Associates Neutrophils, in particular MDSCs, being indicative that the patient is likely to positively respond to a treatment of its cancer by administration of a combination of a) an anti-SIRPα antibody or an antigen-binding fragment thereof that inhibits the binding between SIRPα and CD47, preferably between human SIRPα and human CD47, and b) a compound that is an immune checkpoint inhibitor, in particular a compound that inhibits the binding between PD-1 and PD-L1, preferably between human PD-1 and human PD-L1, in particular an anti-PD-L1 antagonist antibody or an anti-PD1 antagonist antibody, more particularly an anti-PD-1 antagonist antibody or a compound that targets the lymphocyte activation gene-3 (LAG-3), in particular an antibody against lymphocyte activation gene-3.
